SEQUENCE LISTING (1 sequence)

SEQ ID NO: 1 - RNA, 20 nt, synthetic construct
  source 1..20: mol_type = other RNA
ggccacaagttcagcgtgtc

(19) United States
(12) Patent Application Publication
(10) Pub. No.: US 2025/0001000 A1
(43) Pub. Date: Jan. 2, 2025

(54) POLYMERIC NANOPARTICLE FORMULATIONS FOR TARGETED MRNA DELIVERY

(71) Applicant: UNIVERSIDADE DE COIMBRA, COIMBRA (PT)

(72) Inventors: Lino DA SILVA FERREIRA, CONDEIXA-A-VELHA (PT); Artur Filipe CARDOSO DUARTE RODRIGUES, FAJÕES (PT)

(21) Appl. No.: 18/690,528

(22) PCT Filed: Sep. 9, 2022
(86) PCT No.: PCT/IB2022/058516

§ 371 Date: Mar. 8, 2024

(30) Foreign Application Priority Data
Sep. 10, 2021 (PT) 117455

(51) Int. Cl. A61K 47/69; A61K 9/51; C12N 15/88

(52) U.S. Cl. CPC A61K 47/6931; A61K 9/513; C12N 15/88

(57) ABSTRACT

Polymeric nanoparticle formulations based on the combination of 3 monomers were identified lead candidates capable of efficient mRNA delivery, after evaluating 152 formulations by high-throughput screening using a reporter fibroblast model. Using in vitro and in vivo models, this formulation transfected fibroblasts much more effectively than other cell types populating the skin, with superior performance than lipid-based transfection agents in the delivery of Cas9 mRNA and guide RNA. This tropism could be explained by receptor-mediated endocytosis, involving CD26 and FAP, which are overexpressed in profibrotic fibroblasts. Structure-activity analysis revealed that efficient mRNA delivery required the combination of high buffering capacity and low mRNA binding affinity for rapid release upon endosomal escape. These nanoformulations may find multiple applications in the modulation of fibroblasts involved in fibrotic diseases of the skin, heart, liver, and lung, as well as in the microenvironment of epithelial tumors, and in cellular reprogramming.

### TECHNICAL FIELD

This application relates to formulations comprising polymeric nanoparticles for targeted delivery of mRNA to cells.

### BACKGROUND ART

RNA-based therapies have emerged as promising strategies to regulate cell activity in disease and tissue regeneration.^{[1]} Messenger RNA (mRNA)-based therapeutics offer several advantages relatively to plasmid DNA, since mRNA does not require translocation to the nucleus and is more readily biodegradable, thus enabling controlled protein expression without concerns of mutating the genome.^{[2]} Transient protein expression could further expand the therapeutic application of RNA-based therapies to protein replacement and gene editing therapies,^{[2a]} which are expected to emerge in the near future. Progresses have been made in viral and non-viral delivery formulations of mRNA-based therapeutics by maximizing its stability and translation, preventing its immunogenicity and improving its in vivo delivery.^{[2]} However, targeting these formulations to specific cells in the body constitutes an important hurdle in the clinical translation of mRNA-based therapies, because this is critical to decrease their side effects and maximize their efficacy.

Viral delivery systems have shown good efficiency in mRNA delivery but their limited cargo size, immunogenicity. and safety issues related to off-target transfection remain important limitations for their broad use, particularly in gene editing.^{[3]} Lipid and polymeric nanoparticles (NPs) may be an alternative to viral delivery of mRNA, following their extensive demonstration in the delivery of DNA and short interfering RNAs.^{[2b, 4]} These formulations can protect nucleic acids from degradation and prevent unwanted immune responses, and their physicochemical properties can be readily tuned to control their interaction with cells. The recent clinical approval of lipid nanoparticle (LNP) formulations for the delivery of RNA has encouraged the development of NPs for nucleic acid therapies.^{[5]} Yet, their composition can substantially affect its immunogenicity, which is an important aspect considering that inflammatory responses may significantly hamper the translation of mRNA delivered by LNPs.^{[6]} Moreover, therapeutic application of LNPs has been limited due to their preferential uptake by hepatocytes in the liver.^{[7]} In order to address this challenge, LNPs can be functionalized with targeting antibodies to achieve cellular specificity.^{[8]} Although these LNPs have demonstrated therapeutic potential, antibody functionalization requires complex formulation procedures which may deter clinical application.^{[9]} Alternatively, recent studies have demonstrated that the biophysical properties of LNPs can be engineered in order to redirect their biodistribution to other cell types (e.g. endothelial and immune cells) in organs such as spleen, kidney or lung.^{[10]} Although the combination of ionizable and cationic moieties were considered essential to modulate tissue tropism, these efforts have failed to target specific cell types.

Polymeric NPs are also attractive candidates for mRNA delivery owing to their greater chemical versatility, resulting from the combination of different amines and divinyl monomers to generate polymer libraries that can be readily synthesized via Michael-type addition.^{[11]} These libraries have identified chemical features and established design principles for electrostatic complexation and delivery of nucleic acids. In the last 5 years, several polymeric NPs have been reported for the delivery of mRNA encoding luciferase, erythropoietin, tumor suppressing genes, and gene editing enzymes such as Cre recombinase,^{[12]} albeit without specific cell targeting properties. The chemical diversity of combinatorial polymer libraries has been explored to design NP formulations for controlled siRNA delivery in vitro and in vivo to the skin.^{[13]} The influence of polymer chemistry on NP uptake and bioactivity for siRNA delivery encouraged us to further explore these polymers for mRNA delivery. Nevertheless, NPs developed for siRNA delivery may not be suitable for mRNA delivery.^{[14]} While siRNA is typically comprised by two complementary strands of ˜20 nucleotides, mRNA is a single strand spanning up to several kilobases of free nitrogenous bases, which can mediate hydrogen bonding during complexation with cationic NPs, in addition to electrostatic interactions.^{[15]} A combinatorial library of polymers by Michael-type addition of amines and divinyl monomers^{[13, 16]} presenting physicochemical diversity was prepared, in order to identify formulations able to interact more specifically with fibroblasts, without requiring the addition of targeting ligands. Fibroblasts were selected as the cell target due to their involvement in several diseases.^{[17]} These combinations may contain ionizable and cationic moieties to enable electrostatic complexation with nucleic acids and promote endolysosomal escape, while hydrophobic moieties including aromatic compounds improve NP stability in physiological milieu.^{[18]} Functional mRNA delivery was evaluated by high-throughput screening of NP formulations prepared by electrostatic complexation of these polymers with mRNA encoding Cre recombinase, using a fibroblast reporter cell model sensitive to the enzymatic activity of the translated protein. The best polymer candidates were then validated against other skin cell types (keratinocytes, monocytes and endothelial cells), in order to confirm their tropism for fibroblasts. Polymeric NPs were formulated using three different mRNA molecules: (i) one mRNA encoding a fluorescent protein (GFP): and (ii) two mRNAs encoding gene editing enzymes (Cre recombinase and Cas9 nuclease). Cre recombinase has been used for more than 30 years in the generation of transgenic animal models with high relevance in fundamental biology,^{[19]} whereas the CRISPR/Cas9 system has revolutionized gene therapy owing to its precise manipulation of genomes to correct disease-causing mutations.^{[3a]} The performance of the best formulation was then tested regarding its efficacy and cellular specificity using an in vivo model sensitive to Cre recombinase. Finally, the NPs were characterized for their size, surface charge, cellular internalization and protein encoding activity, in order to establish structure-activity relationships and understand which physicochemical properties drive mRNA delivery.

### General Description

The present application relates to s polymeric nanoparticle formulation for targeted delivery of mRNA to cells comprising a polymer made of divinyl monomers bearing an aromatic ring, piperazine-containing divinyl monomers, and amino alcohol monomers, wherein the polymer is complexed with mRNA to form the nanoparticles. The divinyl monomer bearing an aromatic ring can be selected from (2-nitro-1,3-phenylene)bis(methylene) diacrylate or (1,3-phenylene)bis(methylene) diacrylate. The piperazine-containing divinyl monomer can be selected from 1,4-bis(acryloyl)piperazine. The amino alcohol monomers can be selected from 3-amino-1-propanol, 4-amino-1-butanol, 5-amino-1-pentanol, 6-amino-1-hexanol, 7-amino-1-heptanol, 8-amino-1-octanol, 9-amino-1-nonanol, or 10-amino-1-decanol. In one embodiment, the molar ratio per repeating unit of the polymer is 50% divinyl monomers:50% amino alcohol. In one embodiment, the molar ratio per repeating unit of the polymer of any of the divinyl monomers varies between 5% and 45%. In one embodiment the mass ratio of mRNA:polymer is between 1:5 and 1:200. In another embodiment, the mass ratio of mRNA:polymer is 1:100. In one embodiment, the nanoparticles have an average particle size between 200 and 600 nm. In one embodiment, the nanoparticles have a zeta potential between +5 and +25 mV.

The polymeric nanoparticle formulation can be used in the delivery of mRNA to fibroblasts in, but not limited to, skin, heart, liver, lung, synovial joints, or the central nervous system. The polymeric nanoparticle formulation can also be used in the treatment of fibrotic disorders in the skin, liver, lung, or heart; the treatment of autoimmune diseases such as rheumatoid arthritis or Crohn's disease; in anti-ageing treatments or in oncology, through the modulation of fibroblasts to remodel extracellular matrix in the skin and the microenvironment of epithelial tumors; or in cellular reprogramming of fibroblasts in vivo and ex vivo.

### BRIEF DESCRIPTION OF DRAWINGS

FIG. 1. High-throughput screening of polymer library for mRNA delivery. (a) NPs were generated after complexation of mRNA with polymers constituted by a fixed diacrylate moiety (P1) with varying bisacrylamides (A-E) and amines (1-32). Polymer library was screened for the delivery of mRNA encoding Cre recombinase in mouse embryonic fibroblast reporter cell line, expressed by functional activation of GFP at 48 h post transfection. (b) Selected polymers (highlighted in blue after exclusion of false positives) showed comparable transfection efficiency to Lipofectamine 2000. Non-cytotoxic polymers inducing less Cre-mediated recombination than naked Cre mRNA were excluded from this analysis. Data are expressed as mean±SEM (n=3-14). (c) Chemical structures of common bisacrylamides and amines in identified hits. (d) Transfection efficiency of NPs containing mRNA Cre recombinase after polymer purification by dialysis. (d.1) Cre-mediated recombination was quantified by high-content imaging. Data are expressed as mean±SEM (n=6-17). (d.2) Representative fluorescence microscope images of lead NP candidate (P1E28) compared to Lipofectamine 2000. Scale bars=100 μm. Data in (b) and (d.1) were analyzed by one-way ANOVA with post hoc Dunnett's multiple comparisons test against free mRNA and untreated controls. respectively: (*), p<0.05; (**), p<0.01; (***), p<0.001; (****), p<0.0001.

FIG. 2. Hit validation and cellular tropism. (a) Identified polymers were used to transfect GFP mRNA in human dermal fibroblasts. (a.1) Transfection efficiency and (a.2) representative microscope images of human dermal fibroblasts transfected with GFP mRNA. Scale bars=100 μm. (b) Transfection of GFP mRNA in human endothelial cells. (c) human keratinocytes and (d) human monocytes. Data are expressed as mean±SEM (n=3) except for monocytes (n=6). One-way ANOVA with post hoc Tukey's multiple comparisons test was performed: (ns), p>0.05; (*), p<0.05; (***), p<0.001; (****), p<0.0001.

FIG. 3. Development of an RNA-based CRISPR/Cas9 delivery system. (a) CRISPR/Cas9 is based on the delivery of Cas9 (as mRNA) alongside a guide RNA (sgRNA). Gene edition was performed on mouse embryonic fibroblast reporter cell line after Cre-mediated recombination to promote stable GFP expression. Gene edition resulted in decreased GFP expression in transfected cells. (b) Representative images of GFP knockout induced by P1E28 alongside respective controls, obtained at day 3 post transfection using high-content imaging. Scale bars=100 μm. (c) Long-term effects of gene edition quantified at day 10 post transfection using flow cytometry. (c.1) Representative flow cytometry scatter plots of cells treated with P1E28 formulation alongside controls demonstrate GFP knockout. (c.2) Percentage of GFP knockout normalized to the initial GFP expression, assessed by flow cytometry. GFP knockout was calculated after normalizing the number of GFP negative cells in each treatment compared to the untreated control. Data are expressed as mean±SEM (n=3-6). One-way ANOVA with post hoc Dunnett's multiple comparisons test was performed: (*), p<0.05. (d) Cellular uptake of NPs complexed with Cas9 mRNA and a fluorescently labelled sgRNA (ATTO550) by dermal fibroblasts was assessed by flow cytometry 4 h after transfection. (d.1) Percentage of cells internalizing NPs with ATTO550-labelled sgRNA and (d.2) representative scatter plots of cells treated with P1E28 or DharmaFECT Duo. Data are expressed as mean±SEM (n=3). One-way ANOVA with post hoc Tukey's multiple comparisons test was performed: (**). p<0.01. (e) Endosomal disruption induced by P1E28 was measured by quantifying the formation of galectin-9 foci. (e.1) Representative confocal microscopy images of reporter fibroblasts 4 h after incubation with P1E28 or DharmaFECT Duo. Hydroxychloroquine (HCQ) was used as a positive control (60 μM). Scale bars=20 μm (insets=6.5 μm). (e.2) Number of galectin-9 foci per cell. Data are expressed as mean±SEM (n=2-3 independent experiments, with 3-4 confocal images acquired for each replicate). One-way ANOVA with post hoc Tukey's multiple comparisons test was performed: (*), p<0.05; (**), p<0.01.

FIG. 4. In vivo validation of targeted mRNA delivery to skin fibroblasts mediated by P1E28 during wound healing. (a) R26-tdTomato mice were intradermally injected with Cre mRNA delivered by P1E28 or Lipofectamine® 2000. Skin samples were obtained from wounds at day 7 post injection. (a.1) Representative fluorescence microscope images of skin sections illustrate the transfection efficiency of P1E28. generating tdTomato±cells (red) after Cre-mediated recombination. Scale bars=500 μm. (a.2) Quantification of generated tdTomato±cells normalized by tissue section area. Data are expressed as mean±SEM (n=6 animals, 2 wounds per animal). Unpaired two-sample t test was performed: (*), p<0.05. (b) Tropism of P1E28 polyplexes was characterized by immunohistochemical staining of skin sections containing tdTomato±cells. (b.1) Representative fluorescence microscope images illustrate that the vast majority of α-SMA+activated fibroblasts (light blue) co-localized with tdTomato fluorescent areas (red). Scale bars=500 μm. (b.2) Co-localization of each cell type with tdTomato was obtained after calculating the Manders' overlap coefficient. Data are expressed as mean±SEM (n=6 animals, 3-5 images per animal). One-way ANOVA with post hoc Tukey's multiple comparisons test was performed: (****), p<0.0001.

FIG. 5. Structure-activity relationships of polymeric NPs for mRNA delivery. (a) Physicochemical properties of the identified hits: (a.1) surface charge, (a.2) hydrodynamic diameter, and (e.3) complexation efficiency. Results are expressed as mean±SEM (n=3). In (a.1) and (a.2), data were analyzed by one-way ANOVA with post hoc Tukey's multiple comparisons test: (*), p<0.05; (**), p<0.01. In (a.3), data were analyzed by two-way ANOVA with post hoc Sidak's multiple comparisons test: (*), p<0.05. (b) Heparin replacement assay estimated the polymers' binding affinity to Cre mRNA after quantification of fluorescence intensity of bands in each lane corresponding to a different heparin dose. Data represent the mean value of 2-3 gel replicates. Horizontal dashed line corresponds to 50% mRNA release. (c) Acid-base titration for the selected polymers dissolved in 150 mM NaCl, adjusted for pH 3 with HCl. (d) Multiple linear regression of polymer buffering capacity and mRNA binding affinity enabled the correlation of these parameters with transfection efficiency. (d.1) Standard least squares effect leverage with two-way interactions described the significant interaction of these parameters in transfection efficiency, with negative coefficient Heparin*Buffer indicating an inversely proportional relationship. (d.2) Heatmap representation of the predicted model with highlighted experimental points corresponding to selected polymers.

FIG. 6. Molecular mechanism of P1E28 formulation for fibroblast targeting. (a) Size of polyplexes generated by complexation of P1E28 with Cre mRNA. (a.1) Representative TEM image and (a.2) NP diameter distribution show the formation of small NPs (87% of measured NPs<200 nm). Scale bar=500 nm. (b) Recombination in mouse reporter fibroblast cell model after endocytosis inhibition of P1E28 via clathrin-(chlorpromazine) and caveolin-mediated endocytosis (filipin III), as well as actin-related processes (cytochalasin D). Data are expressed as mean±SEM (n=5-6). One-way ANOVA with post hoc Dunnett's multiple comparisons test was performed: (**), p<0.01; (***), p<0.001; (****), p<0.0001. (c) Transfection efficiency was determined by quantifying the number of cells expressing GFP, after delivery of GFP mRNA to human dermal fibroblasts with or without receptor blocking using monoclonal antibodies targeting CD26 and FAP. Data are expressed as mean±SEM (n=3). Two-way ANOVA with post hoc Sidak's multiple comparisons test was performed: (*), p<0.05; (**), p<0.01. (d) Impact of receptor blocking on cellular uptake of NPs complexed with Cas9 mRNA and a fluorescently labelled sgRNA (ATTO550) was assessed by flow cytometry. (d.1) Percentage of cells internalizing NPs with ATTO550-labelled sgRNA 4 h after transfection and (d.2) representative scatter plots of cells treated with P1E28 with or without antibody blocking. Data are expressed as mean±SEM (n=3). Two-way ANOVA with post hoc Sidak's multiple comparisons test was performed: (**), p<0.01; (****), p<0.0001.

FIG. 7. Schematic representation of monomers used to synthesize polymer library. Polymers were generated after combining P1 diacrylate with bisacry lamides (A-E) and amines (1-32).

FIG. 8. High-throughput screening of polymer library for mRNA delivery. (a) Screening strategy of the polymer library for the delivery of mRNA encoding Cre recombinase. (b) Heatmap representation of fibroblast cytotoxicity (based on nuclear condensation) of polymeric NPs, illustrates the impact of the amine monomer, whereas the bisacrylamide had no effect on cell viability. (c) Transfection efficiency resulting in the expression of GFP upon Cre-mediated recombination was validated after careful analysis of microscopy images, which showed some false positives. Scale bars=50 μm. The best true positives were highlighted with blue bars in FIG. 1b.

FIG. 9. Cytotoxicity of top-performing polymers and NP formulations in fibroblasts. The polymers were purified by dialysis in DMSO using regenerated cellulose membranes (MWCO=2 kDa). (a) Fibroblasts were incubated with the purified polymers alone at different doses to establish toxicological dose-response relationships by PI staining. (b) Toxicological dose-response curves of polymeric NPs (formulated by complexation with a fixed dose of 50 ng mRNA encoding GFP) supported polyplex optimization using different mRNA:polymer mass ratios. Selected ratios were highlighted in dark yellow, considering a cytotoxicity threshold at 50%. similarly as FIG. 8. Data are expressed as mean±SEM (n=3).

FIG. 10. Optimization of polyplexes for co-delivery of Cas9 mRNA and GFP sgRNA. (a) Agarose gel electrophoresis of P1E28 complexed with (a.1) Cas9 mRNA and (a.2) GFP sgRNA determined optimal mass ratios to yield stable complexes. (b) Optimization of Cas9 mRNA and GFP sgRNA doses for co-delivery using (b.1) commercially available transfection agents and (b.2) the lead polymer candidates. First, mRNA and sgRNA doses were adjusted using the commercial agents by testing different mRNA:sgRNA mass ratios from 1:0.25 to 1:2. The selected doses for commercial agents were 100 ng mRNA±100 ng sgRNA (mRNA:sgRNA ratio=1:1). In the case of the tested polymers, the optimal sgRNA dose was 25 ng for 100 ng mRNA (mRNA:sgRNA=1:0.25), indicating a greater efficiency than lipid-based agents in the delivery of RNA molecules for gene editing. Polymers were complexed at mRNA:polymer mass ratio of 1:10, except PIA05 due to its toxicity (1:2). Two-way ANOVA was performed with post hoc Tukey's multiple comparisons test: (*), p<0.05; (**), p<0.01; (****), p<0.0001. (c) Optimization of polymer dose regarding transfection efficiency and cytotoxicity, using a fixed mRNA dose of 100 ng and mRNA:sgRNA ratio=1:0.25. Selected mRNA:polymer ratios are highlighted in dark yellow, after exclusion of cytotoxic ratios (considering threshold at 50% in dashed line) and excessive polymer doses resulting in erroneous identification of GFP-negative cells due to autofluorescence (e.g. P1C05). (d) Comparison of co-delivery of Cas9 mRNA (100 ng) and sgRNA (25 ng) with staged delivery using Lipofectamine® 2000 to transfect sgRNA. Dashed line corresponds to abundance of GFP-negative cells in the untreated control. No significant differences among delivery strategies were detected after performing two-way ANOVA with post hoc Tukey's multiple comparisons test. Data in (b), (c), and (d) are expressed as mean±SEM (n=2-4).

FIG. 11. Cellular uptake of P1E28 complexed with Cas9 mRNA. Internalization of P1E28 complexed with Cas9mRNA was compared to the commercial agent DharmaFECT Duo using an ATTO550-labelled sgRNA. (a) Representative fluorescence images show an increased ATTO550 signal in human dermal fibroblasts treated with P1E28 compared to DharmaFECT Duo. Scale bars=50 μm. (b) Total fluorescence intensity of ATTO550 was normalized by the number of cells in each field. One-way ANOVA with post hoc Tukey multiple comparisons test was performed: (*), p<0.05. Data are expressed as mean±SEM (n=3 independent measurements, each corresponding to the average of 7 images).

FIG. 12. Wound healing process over time. Wound closure was quantified by comparing the reduction of wound area over time. Data are expressed as mean±SEM (n=2-6).

FIG. 13. Immunohistochemical staining of skin sections. Representative fluorescence microscopy images monitoring co-localization of tdTomato signal with (a) CD31, (b) CD68, (c) keratin 14 enabled the identification of transfected endothelial cells, macrophages, and keratinocytes, respectively. Scale bars=50 μm.

FIG. 14. Polyplex stability in water and cell culture medium. Purified polymers were complexed with Cre mRNA at the optimized mRNA:polymer mass ratios and diluted in water or cell culture medium (with or without 10% FBS). Polyplex stability (hydrodynamic diameter and polydispersity index, PDI) was measured by DLS in water (for 96 h) and cell culture medium (for 4 h) for lead candidates (a) P1A04, (b) P1A05, (c) P1C05, (d) P1C06, (e) P1E04 and (f) P1E28. Data are expressed as mean±SEM (n=5).

FIG. 15. Influence of mRNA on polyplex physicochemical properties. (a) Normalized buffering capacity of each polymer was determined from the titration curves in FIG. 5c. All polymers showed a clear effective pKa between pH 4-5, with weaker ionization steps at more basic pH (7-9). (b) Binding affinity of polymers complexed with GFP mRNA was estimated by heparin replacement assay, (b.1) showing similar profiles to those obtained with Cre mRNA (FIG. 5b). (b.2) Estimated amount of heparin required to release 50% of complexed mRNA (EC50) was not significantly different between GFP and Cre mRNA (p>0.05). after performing a two-way ANOVA with post hoc Tukey's multiple comparisons test. Results are expressed as mean±SEM (n=2-3). (c) Complexation efficiency of GFP mRNA was assessed by SYBR Gold labelling, (c.1) evidencing a similar pH-dependent behavior for all polymers. (c.2) Binding affinity toward GFP mRNA was compared with Cre mRNA (FIG. 5a). Although the selected polymers seemed to complex GFP mRNA less efficiently than Cre mRNA at pH 7.5, this was only significant for PIC05. Results are expressed as mean±SEM (n=3). Two-way ANOVA was performed with post hoc Tukey's multiple comparisons test: (***), p<0.001. In (c.1). statistically significant differences between groups are indicated by different letters (p<0.05). (d) P1E28 was complexed with GFP mRNA or Cas9 mRNA+GFP sgRNA for structural characterization. (d.1) Representative TEM images and (d.2) size distribution of P1E28 complexed with different mRNAs illustrated larger polyplex size when the polymer is complexed with Cas9 mRNA. Bars in (d.2) correspond to mean±SEM of >100 measured NPs: Cre=130.9±22.2 nm; GFP=176.7±11.9 nm; Cas9=256.8±26.7 nm. One-way ANOVA test was performed with post hoc Tukey's multiple comparisons test: (**), p<0.01; (***), p<0.001. (d.3) Binding affinity determined by heparin replacement assay showed significantly higher affinity of P1E28 towards Cas9 mRNA and GFP sgRNA. Results are expressed as mean±SEM (n=2-3). A Kruskal-Wallis test was performed with post hoc Dunn's multiple comparisons test: (*), p<0.05.

FIG. 16. Lack of correlation between transfection efficiency and physicochemical properties of polyplexes. Data points represent the mean values of: (a) hydrodynamic diameter, and (b) ξpotential, obtained by DLS; (c) particle number obtained by NTA; (d) complexation efficiency determined by SYBR Gold fluorescence assay; (e) binding affinity, represented by the dose of heparin required to induce 50% of mRNA release from polyplexes (EC50); (f) buffering capacity corresponding to the amount of NaOH required to increase solution pH from 5 to 7.5 (in μmol mg⁻¹ polymer). Pearson correlation test showed no significant association of these individual parameters with transfection efficiency (p>0.05).

FIG. 17. Multiple linear regression of polymer characteristics in transfection efficiency. (a) Transfection efficiency of the selected polymers was correlated simultaneously with their (a.1) mRNA binding affinity and (a.2) buffering capacity. Data were fitted to a standard least squares model, set to effect leverage. (a.3) First-order interactions between these parameters were also considered, given that P1E28 exhibited the highest buffering capacity, which is associated with successful endosomal release, and the lowest mRNA binding affinity, comparable to Lipofectamine® 2000 (FIG. 5b-c). Both parameters were shown to interact negatively with each other and affect transfection efficiency (p=0.0035). (b) Predicted model corresponding to FIG. 5d.2, highlighting P1E28 and PIC06, which was not plotted in the main figure because it exhibited an exceptionally high mRNA binding affinity. For fixed transfection efficiency values ranging from 0 to 100, the generated model predicted that high biological activity could be attained by designing polymers with: i) low mRNA binding affinity (EC50<100 μg heparin μg⁻¹ mRNA) and high buffering capacity (>30 μmol NaOH mg⁻¹ polymer); ii) high mRNA binding affinity (EC50>100 μg heparin μg⁻¹ mRNA) and low buffering capacity (<20 μmol NaOH mg⁻¹ polymer).

FIG. 18. Comparison of transfection efficiency among polymers with similar chemical features. (a) Cre mRNA was complexed with non-purified polymers sharing the same amine 28, resulting in negligible activity compared to P1E28. Some polyplexes exhibited lower recombination rates than naked mRNA (‡). (b) Purified P1E28 transfected Cre mRNA more effectively than other similar polymers, sharing the same bisacry lamide and diacry late moieties but varying amine group by the length of the alkyl chain. Although purified polymer doses were adjusted with the aim of optimising transfection efficiency, none of the tested polymers could outperform P1E28. (c) Transfection efficiency of P1E28 required the presence of the diacry late moiety P1. Data are expressed as mean±SEM (n=3).

FIG. 19. Characterization of CD26/DPP4 family receptors in fibroblasts. (a) Representative immunofluorescence images of human dermal fibroblasts (AG08468) and mouse reporter fibroblast model used for screening the polymer library (SFr) stained for CD26 (DPP4) and FAP. Scale bars=30 μm. (b) Total fluorescence intensity of each protein was normalized by cell area in each field. Two-way ANOVA with post hoc Sidak multiple comparisons test was performed: (****), p<0.0001. Data are expressed as mean±SEM (n=5 images obtained from 2 independent measurements per condition).

FIG. 20. List of monomers used to synthesize polymer library.

### DETAILED DESCRIPTION OF EMBODIMENTS

### 2.1. High-Throughput Screening of Polymer Library

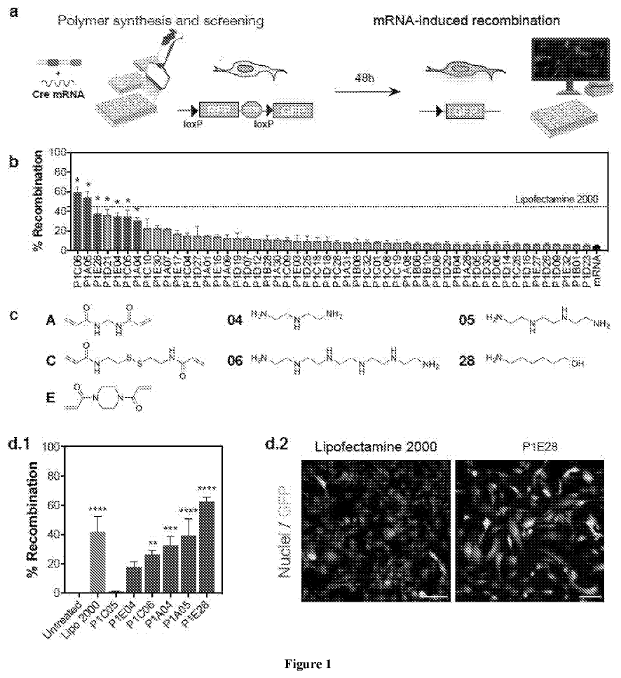

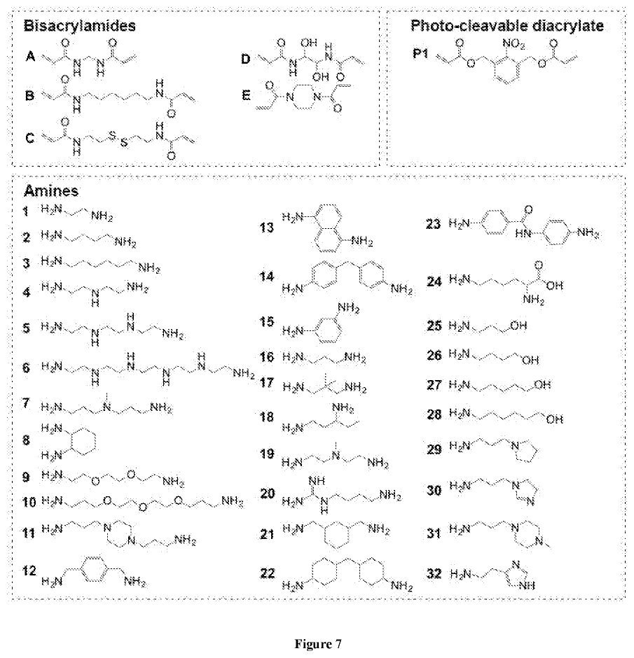

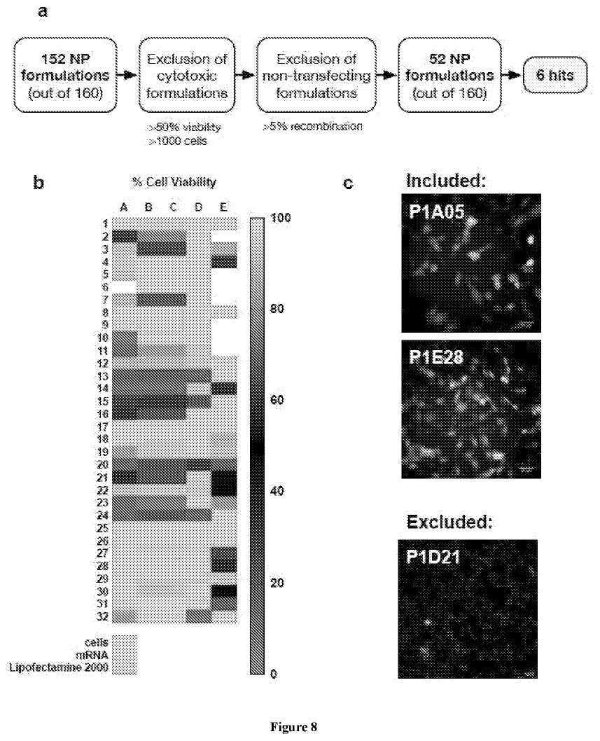

The polymer library was synthesized via Michael-type addition of a fixed hydrophobic diacry late (P1) with chemically diverse bisacrylamides (A-E) and amines (1-32), dispersed in dimethyl sulfoxide (DMSO) at a molar ratio of 1:1:2.respectively. Chemical structures of the monomers used in this study are described in FIG. 7. Finally, these terpolymers were end-capped with an excess 10% molar ratio of the respective amines, in order to improve polymer biocompatibility and transfection efficiency.^{[13a, 20]} This synthetic approach enabled the rapid generation of 152 polymers out of 160 possible combinations for complexation with mRNA, with the remaining 8 combinations unable to be formed due to gelation. NP formulations were prepared in nuclease-free water by mixing each newly synthesized polymer (dissolved in DMSO) with mRNA encoding Cre recombinase at a fixed mass ratio of 100:1, respectively. High-throughput screening was performed in 96-well plates by treating a mouse embryonic fibroblast reporter cell line with polymeric NPs (50 ng/well Cre mRNA) dispersed in serum-free cell culture medium. Lipofectamine 2000 was used as a positive control following its demonstration of effective mRNA delivery in different cell models. Successful mRNA delivery to these cells resulted in the expression of Cre recombinase, which excises the loxP-flanked STOP cassette and ultimately enables gene expression of GFP (FIG. 1a).^{[22]} High-content imaging enabled simultaneous analysis of transfection efficiency and cytotoxicity (FIG. 8). Most formulations were well-tolerated by the reporter fibroblasts, despite the presence of residual DMSO (<1% v/v). Although this solvent has been employed in previous screening studies of polymeric NPs and showed negligible toxicity at the concentrations used herein,^{[23]} these polymers can also be prepared in aqueous solutions prior to complexation with mRNA,^{[24]} in order to facilitate their clinical translation. From the 52 non-cytotoxic formulations with superior transfection efficiency than naked mRNA alone (FIG. 1b), 2 polymers (P1C06, P1A05) induced comparable gene recombination to commercial Lipofectamine 2000, followed by 4 other hits (P1E28, P1E04, P1C05, P1A04) which also induced significant GFP expression compared to vector-free mRNA treatment (p<0.012). P1D21 (p=0.0397) was excluded after visual inspection of microscopy images because the quantified green fluorescence was due to polymer autofluorescence rather than GFP expression after transfecting with Cre mRNA (FIG. 8c). Hence. we selected 6 polymers that shared chemical features such as the incorporation of linear alkyl amines 4, 5, or 6, differing primarily by the combination with bisacrylamides A, C, or E (FIG. 1c). These alkyl amines are readily protonated at physiological pH, thus conferring high cationic charge density, which may improve electrostatic complexation of mRNA and NP uptake.^{[2b]} P1E28 strikingly contrasted from the remaining polymers with the incorporation of an alkyl alcohol side chain combined with the presence of bisacry lamide E, which is characterized by piperazine rings containing two tertiary amines conferring the polymer with high buffering capacity.^{[11d, 25]}

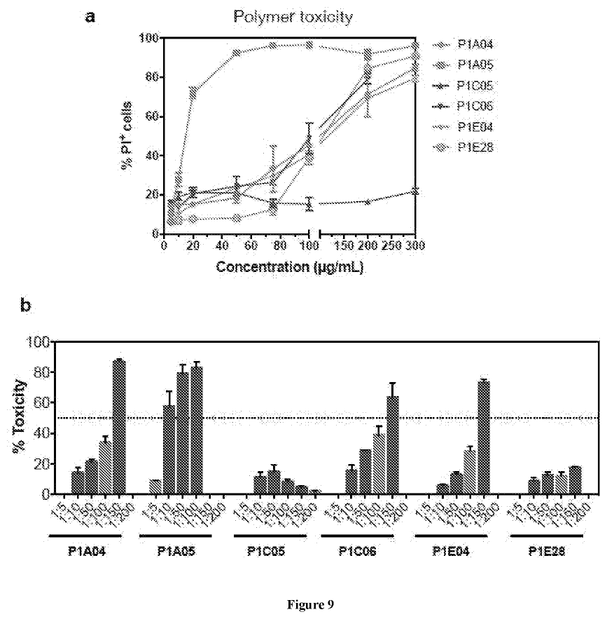

Activity of the selected polymers was further validated after purification by dialysis in DMSO (MWCO=2 kDa), in order to remove free monomers and short polymer chains that might compete for electrostatic complexation with mRNA and thus reduce the number of functional polyplexes. The mRNA:polymer mass ratio of the polymeric nanoformulations was adjusted accordingly to minimize cytotoxicity (FIG. 9). Interestingly, P1E28 polymer emerged as the lead candidate (FIG. 1d.1), inducing superior cell recombination in fibroblasts (FIG. 1d.2) than Lipofectamine 2000 (62.1±3.3% vs 45.1±10.4%), followed by P1A05 (39.0±11.7%) and P1A04 (32.5±6.2%) which induced comparable gene recombination. The performance of P1E28 was not sensitive to batch-to-batch variation (p=0.4611, n=4 batches, data not shown).

### 2.2. Hit Validation Against Several Human Cell Types Using Different mRNA Molecules

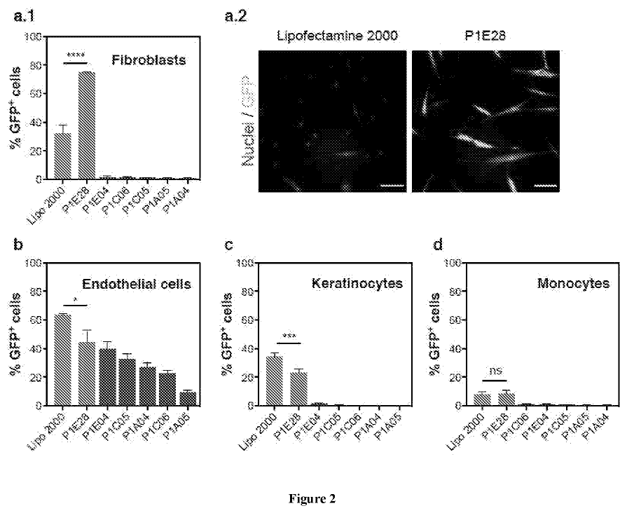

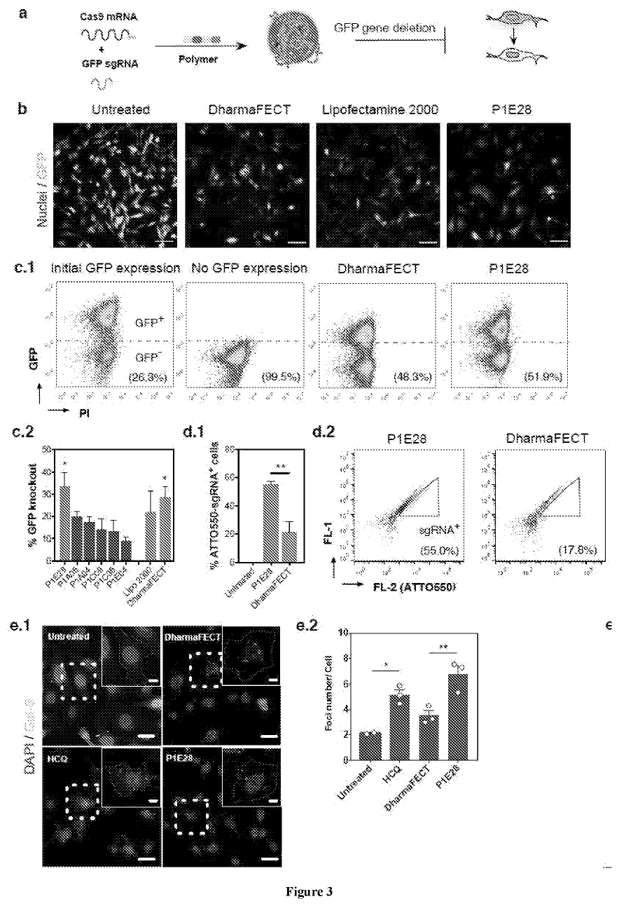

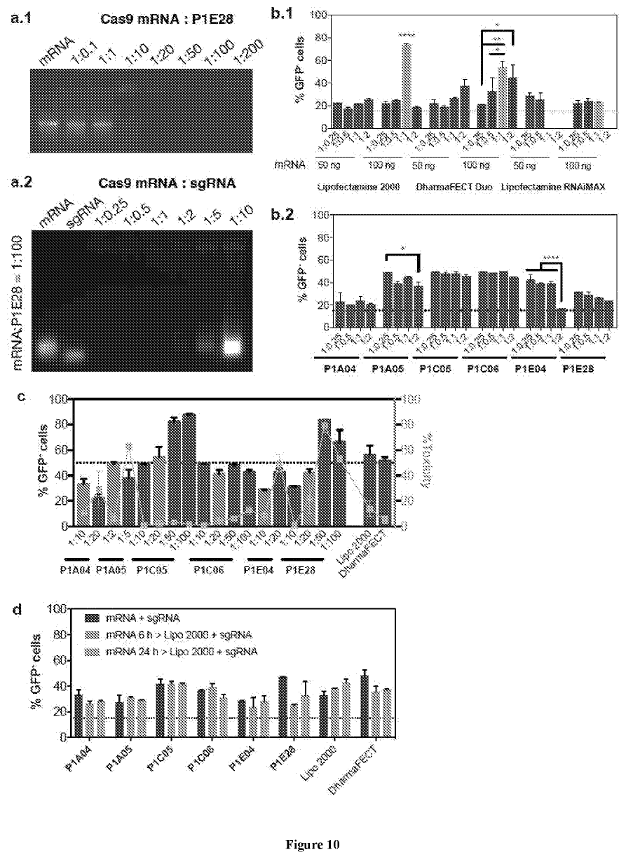

The hit formulations were then evaluated across several human cell types using a second mRNA, in this case encoding GFP protein. Because the mouse fibroblast reporter cell line used here is highly sensitive to the transfection of mRNA encoding Cre recombinase, GFP mRNA validated the polymer efficacy to deliver quantifiable amounts of mRNA functionally expressing protein, based on its fluorescence. P1E28 exhibited superior performance in inducing GFP expression than the remaining polymer candidates across the 4 tested cell types (FIG. 2), albeit only in human dermal fibroblasts (74.5±1.6%) this formulation outperformed Lipofectamine 2000 (p<0.0001). Notably, P1E28transfected human dermal fibroblasts more efficiently than endothelial cells (44.5±8.6%), keratinocytes (23.4±2.2%), and monocytes (8.2±2.7%), which suggested its preferential tropism towards fibroblasts. The remaining hits failed to achieve significant transfection efficiency levels across cell types except in endothelial cells. The validated hits were further investigated in the context of gene editing. In addition to Cre recombinase, the CRISPR/Cas9 system has emerged as a promising tool for precise genome modifications, as it uses tunable sgRNAs to direct the enzyme to any target site in genomic DNA.^{[3a]} Hence, CRISPR/Cas9 systems based on RNA delivery require the delivery of both mRNA and sgRNA, which have significant differences in size (molecular weight) and charge density (FIG. 3a). Because mRNA encoding Cas9 is much longer (4 kb) than Cre mRNA and GFP mRNA (approximately 1 kb), this required further optimization of the delivery system, namely the doses of vector, mRNA and sgRNA (FIG. 10). In order to compare transfection performance of gene editing tools (CRISPR/Cas9 and Cre recombinase) in the same cell type, the reporter fibroblasts were transfected with Cre recombinase protein to stably express GFP in approximately 80% of the treated cells. Functional delivery of Cas9 mRNA and sgRNA targeting GFP was evaluated by high-content imaging, 72 h after transfection (FIG. 3b), in order to assess the knockout of GFP fluorescence after gene editing by non-homologous end joining repair upon DNA excision. As positive controls, we have used 3 different commercial agents (Lipofectamine 2000, Lipofectamine RNAiMAX, DharmaFECT Duo), with variable capacity to load nucleic acids with different sizes, to deliver both Cas9 mRNA and sgRNA (FIG. 10b).

Lipofectamine 2000 and DharmaFECT Duo were superior than Lipofectamine RNAiMAX at the optimal formulation using mRNA:sgRNA ratio=1:1. Polymer candidates achieved optimal transfection using 4 times less sgRNA than lipid-based vectors to complex with the same dose of mRNA, resulting in a ratio mRNA:sgRNA=1:0.25 (FIG. 10b-c). Furthermore, separate delivery of mRNA and sgRNA using different vectors did not offer any benefit to the overall transfection (FIG. 10d), hence we proceeded with the complexation of both nucleic acids in the same vector to ensure they both reach the same target cell.^{[7b]}

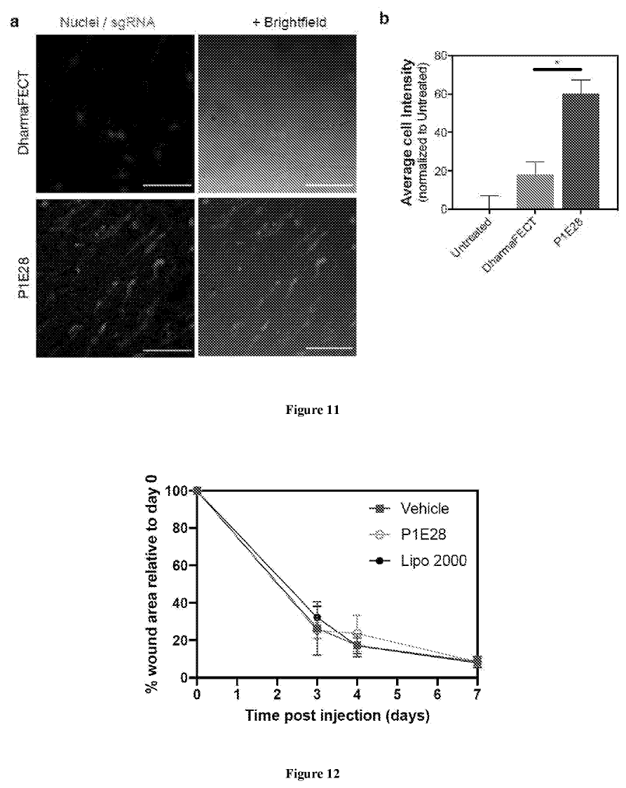

Long-term efficacy of CRISPR/Cas9 gene edition was evaluated by flow cytometry at day 10 post transfection, in order to validate initial results demonstrating decreased fluorescence after excision of GFP induced by Cas9 (FIG. 3c.1).^{[26]} All formulations were shown to have a positive effect in the reduction of GFP fluorescence, indicating that genomic edition of GFP was performed. Nevertheless, only P1E28 (33.4±6.5%, p=0.0112) and DharmaFECT Duo (28.4±5.1%, p=0.0407) significantly increased the abundance of GFP-negative cells compared to the untreated control (FIG. 3c.2). In line with previous findings using mRNA encoding Cre recombinase and GFP, P1E28 emerged as the top performing candidate for co-delivery of Cas9 mRNA and sgRNA. This formulation was more efficient than DharmaFECT Duo because it required 4 times less sgRNA for the same mRNA dose to achieve comparable GFP knockout. This could be explained by the superior cellular uptake of P1E28 polyplexes. When formulated with the same amounts of Cas9 mRNA and a labelled GFP sgRNA, cells treated with P1E28 exhibited an average fluorescence intensity 3.4 times higher (p=0.0145) compared to DharmaFECT Duo (FIG. 11), suggesting efficient polyplex uptake by dermal fibroblasts. These findings were further demonstrated by flow cytometry (FIG. 3d), showing that 55.0±2.4% of the cells effectively internalized P1E28, compared to only 21.5±7.2% with DharmaFECT Duo (p=0.0099).

Effective mRNA delivery requires not only that the nanoformulation is taken up by cells but it also escapes the endosomal compartment and releases its cargo to the cytosol. Intracellular trafficking studies were performed to further determine how P1E28 compares with commercial transfection agents. The induction of galectin-9 foci after NP internalization was monitored to determine the capacity of P1E28 to escape the endosomal compartment (FIG. 3e). This protein has been reported to sense endosomal vesicle disruption.^{[27]} Cells treated with P1E28 showed an increased number of galectin-9 foci compared to those treated with DharmaFECT Duo (p=0.0090). These results were comparable to the treatment with hydroxychloroquine (HCQ), which was used as a positive control (p=0.0221 vs negative control), suggesting that P1E28 could rapidly escape from the endosomal compartment within 4 h after transfection.

### 2.3. In Vivo Validation of Lead Polymeric Formulation During Acute Wound Healing in the Skin

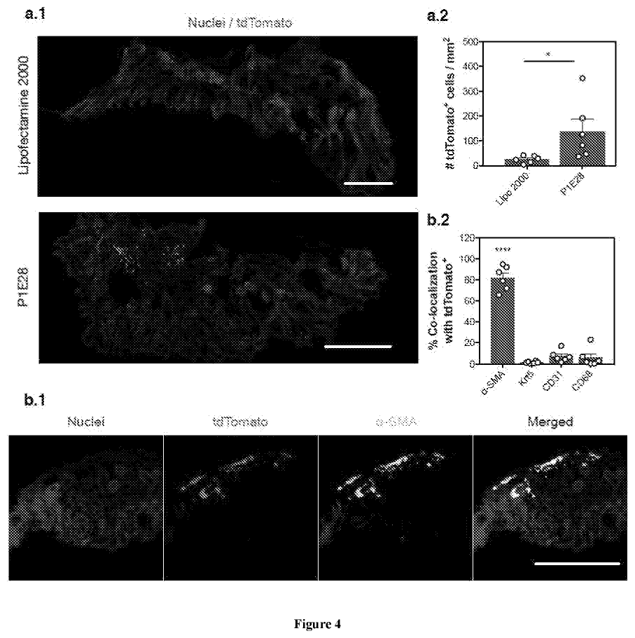

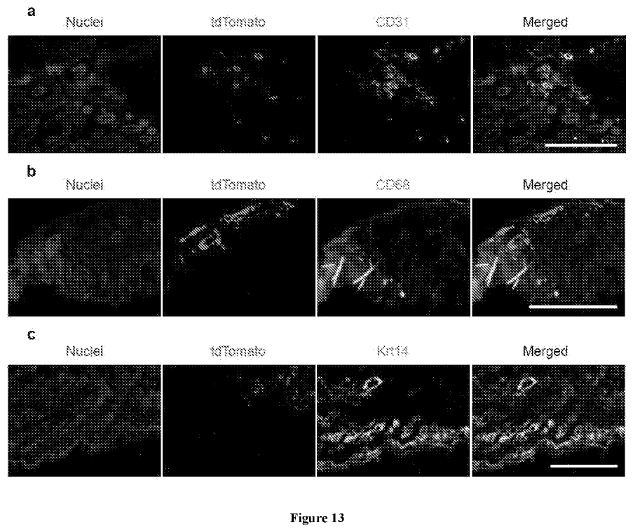

The improved performance and selectivity of P1E28 in the delivery of mRNA to skin fibroblasts (FIG. 2) compared to lipid-based transfection agents was validated in vivo, in the context of acute wound healing (FIG. 4). Polymeric NPs complexed with Cre mRNA were injected intradermally around the wound (4 different locations) in R26-tdTomato mice at day 5 post wounding, when the granulation tissue is established to restore blood supply and to promote regeneration of the skin epithelium. Similarly to SFr cells used in this screening approach (FIG. 1a). this model contains a loxP-flanked STOP cassette which can be excised by Cre recombinase, resulting in the expression of fluorescent tdTomato (FIG. 4a.1). Skin sections were harvested 7 days post injection to allow for the Cre-mediated recombination process to occur. The administered nanoformulations had no impact in physiological wound closure (FIG. 12). P1E28 elicited the recombination of approximately 5.5 times more tdTomato fluorescent cells (139.2±48.9 cells mm⁻² vs 25.3±9.5 cells mm⁻², p=0.0419) than Lipofectamine 2000 (FIG. 4a.2). These regions confirming effective mRNA delivery were further analyzed by immunohistochemistry to characterize cell targets of P1E28 polyplexes (FIG. 4b.1 and FIG. 13). The tdTomato signal evidencing areas of Cre-mediated recombination was strongly co-localized with activated fibroblasts (82.0±4.8%), which typically express α-smooth muscle actin (α-SMA) in acute skin wounds (FIG. 4b.2).^{[}17] In contrast, endothelial cells (CD31+, 7.4±2.6%). macrophages (CD68+, 5.9±3.4%), and keratinocytes (Krt5+, 1.6±0.7%) were poorly co-localized with tdTomato, supporting the preferential tropism of P1E28 towards fibroblasts (p<0.0001) as observed in vitro.

### 2.4. Structure-Activity Relationships Predict Efficacy of mRNA Delivery

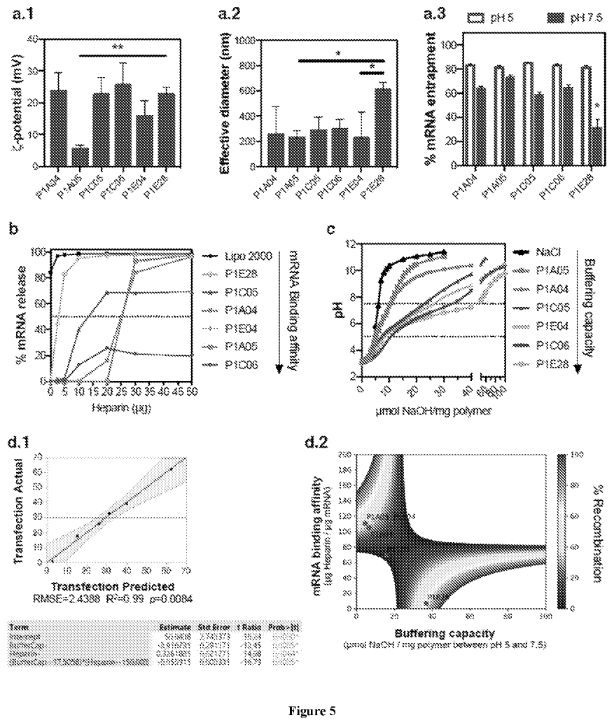

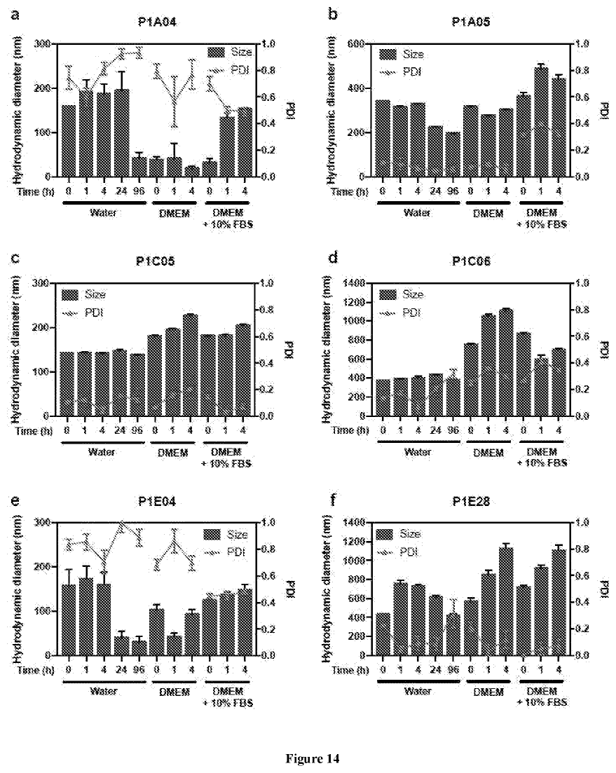

In order to understand the differences among the identified hits with respect to their cellular activity, we characterized polymeric NPs prepared by electrostatic complexation with Cre mRNA (FIG. 5). All formulations rendered positively charged NPs, with-potential values ranging between +5 and +25 mV (FIG. 5a.1) and average particle sizes between 200 and 600 nm (FIG. 5a.2). All polymers except P1A05 revealed a significantly greater complexation efficiency at pH 5 compared to pH 7.5 (p<0.0017), which suggested the predominance of electrostatic interactions ensuring complexation efficiencies around 60% at physiologically relevant pH 7.5 (FIG. 5a.3). This could be attributed to the presence of primary amines both in the side chains and end-caps. Interestingly, despite its high transfection efficiency, the complexation efficiency of P1E28 was the lowest among the selected polymers, and decreased from 80.9±2.2% at pH 5 to 30.8±7.4% at pH 7.5. Nevertheless, all polyplexes maintained their colloidal stability in nuclease-free water for at least 96 h, and in serum-free medium used to transfect the reporter fibroblasts for 4 h (FIG. 14). Supplementation of cell culture medium with serum proteins (FBS) did not significantly affect the colloidal stability of NP formulations.

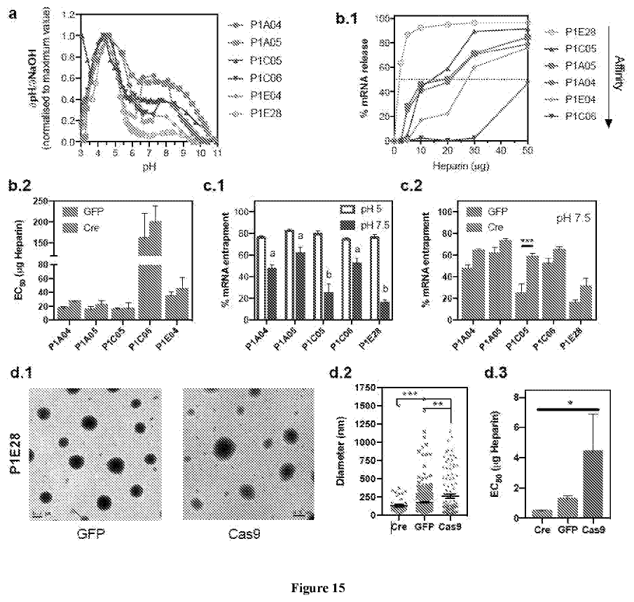

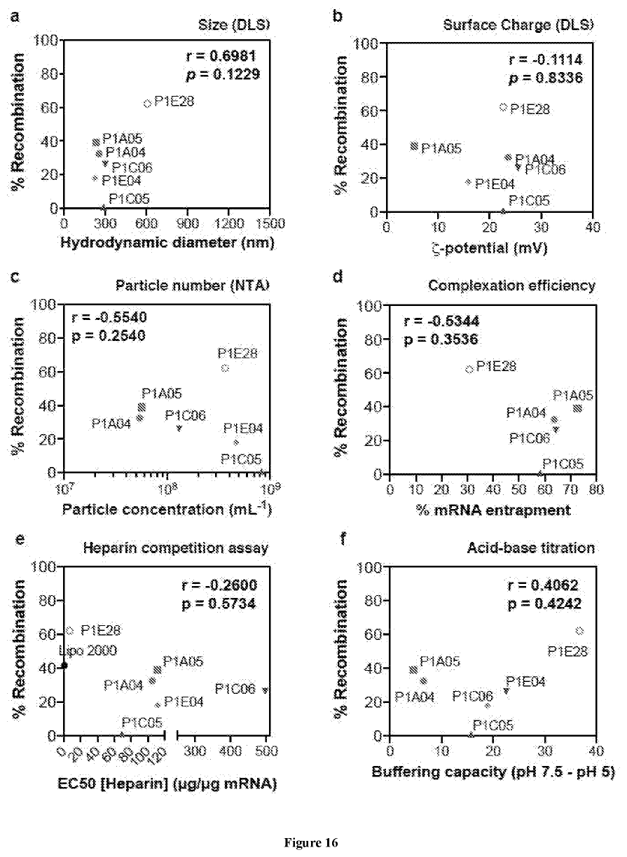

The affinity of mRNA to the polymeric formulations was measured by a heparin replacement assay, which consists in the destabilization of electrostatic interactions in the presence of competing negative charge (FIG. 5b). P1E28 and Lipofectamine 2000 presented extremely low binding affinity towards mRNA, suggesting that this polymer could readily release its cargo. Binding affinity and particle size were not significantly affected by the mRNA cargo (FIG. 15), despite differences between Cre recombinase and GFP mRNA in length (1350 vs 996 nucleotides) and GC content in the coding sequence (68.8% vs 61.5%), which influence the formation of secondary structures and thus may affect how mRNA is complexed with these polymers. Nonetheless, the selected polymers seemed to have lower complexation efficiency of GFP mRNA compared to Cre mRNA (FIG. 15), even though this was only statistically significant for P1C05 (p=0.0004). Notably, P1E28 had the lowest complexation efficiency among the identified hits. Endosomal escape of polymeric NPs is highly correlated with the polymer buffering capacity to overcome vesicle acidification.^{[11d]} Acid-base titration showed that the selected polymers are capable of proton buffering in physiological pH (FIG. 5c), with estimated pKa values between and 4.1 and 4.8 (FIG. 15). Among all polymers, P1E28 was the one that buffered the most protons between pH 5 and pH 7.5, corresponding to a buffering capacity of 36.8 μmol H⁺ mg⁻¹ polymer. The remarkably low binding affinity of mRNA to P1E28 and the polymer's high buffering capacity led to the hypothesis that both factors determined its efficacy towards mRNA intracellular delivery, considering that no correlation between biological activity (i.e. Cre-mediated recombination) and these factors was observed for the remaining polymers (FIG. 16). Similarly, polymer physicochemical properties such as particle number, size or surface charge were poor predictors of biological activity.

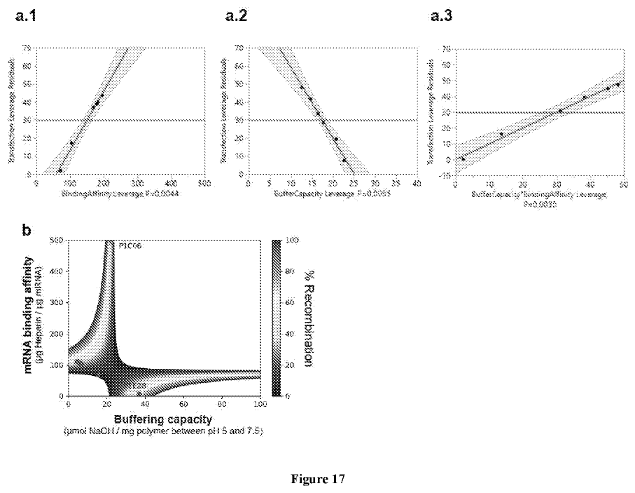

Hence, it was possible that NP activity could not be predicted by a sole factor, but rather the combination of these parameters. For this purpose, multiple linear regression using a standard least squares model was performed to assess whether mRNA binding affinity and polymer buffering capacity played a role in NP activity (FIG. 17). A statistically significant model was obtained (p=0.0084) describing that both parameters negatively affected each other and determined the formulation's performance (FIG. 5d.1 and FIG. 17). The design space generated from the experimental results consisted of two major categories (FIG. 5d.2): (i) polymers with low buffering capacity (<20 μmol NaOH mg⁻¹ polymer) and high mRNA binding affinity (>100 μg heparin μg⁻¹ mRNA); or (ii) polymers with high buffering capacity (>30 μmol NaOH mg⁻¹ polymer) and low mRNA binding affinity (<100 μg heparin μg⁻¹ mRNA). The first category corresponds to polymers constituted by alkyl amines, which confer a greater cationic content that enables the uptake of greater amounts of complexed mRNA (FIG. 5a.3). However, their low buffering capacity may preclude their release from acidified endosomes, leading to their degradation. Here, the selected cationic polymers performed best in endothelial cells (FIG. 2b), where they could exploit alternative endocytic mechanisms such as caveolae-mediated pathways,^{[31]} which target instead the Golgi and the endoplasmic reticulum.^{[32]} Further investigation on the influence of endocytic pathways in mRNA delivery to each cell type is warranted to maximize transfection efficiency. Another aspect that merits further consideration is the fact that a high binding affinity may be detrimental for mRNA release to the cytosol. In order to circumvent that issue, cationic polymers can be designed with shorter chains to weaken their electrostatic interactions with mRNA.^{[33]}

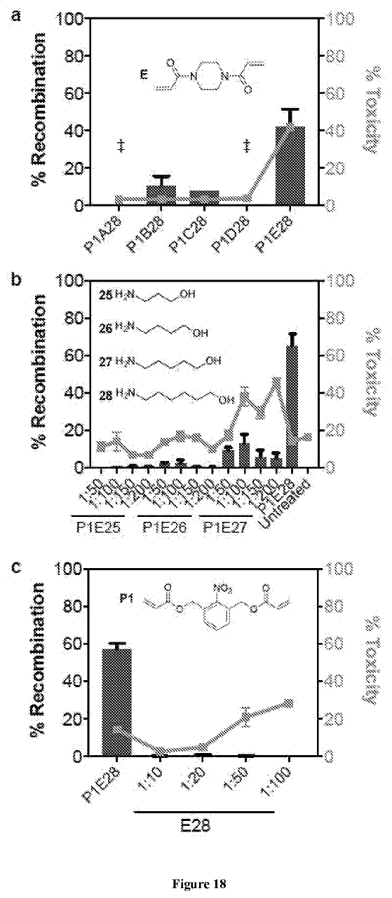

In contrast with the remaining polymers, P1E28 fits in the latter category representing an ideal candidate to overcome some of the major challenges in polymer-based vectors: i) entrapment in the endolysosomal compartment, ii) dissociation and release of mRNA to the cytosol. While its low binding affinity was comparable to commercially available transfection agents (FIG. 5b) and is associated with efficient mRNA release for protein translation.^{[21a, 34]} P1E28 may benefit from its high buffering capacity to promote endosomal escape to the cytosol. The capacity of P1E28 to destabilize the endosomal compartment (FIG. 3e) can also be explained by its strong ionization at endosomal pH,^{[30b]} as suggested by the sharp difference in mRNA complexation between pH 7.4 and pH 5 (FIG. 5a.3). These properties seemed to be dictated by the unique combination of the 3 different monomers constituting the polymer (FIG. 18), as transfection efficiency was abrogated when the hydrophobic diacrylate (P1) was removed and the bisacry lamide moiety (E) replaced by the other molecules used to synthesize the library. Interestingly, the length of the alkyl chain of the amino alcohol moiety (28) also determined polymer performance, with longer chains favoring transfection. While hydrophobicity may be an important feature determining polymer stability, we hypothesized that these chemical features could also determine the apparent selectivity of P1E28 toward fibroblasts (FIG. 3d).

### 2.5. Proposed Molecular Mechanism Behind the NP Targeting to Fibroblasts

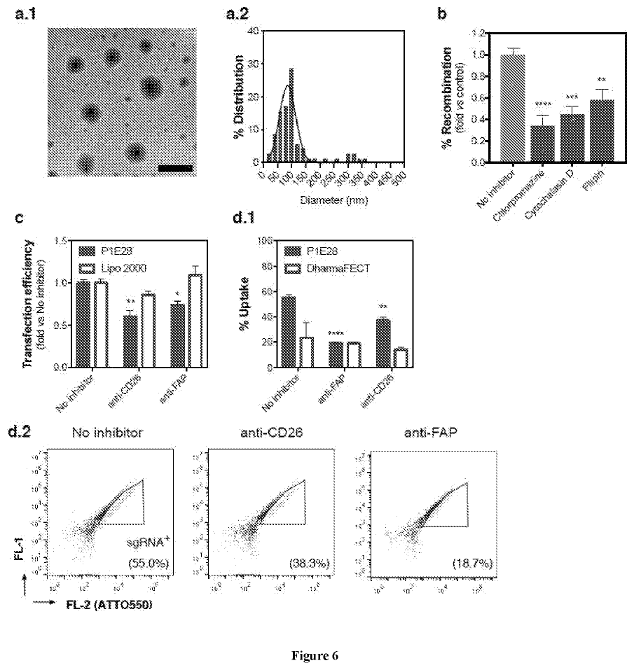

P1E28 polyplexes were characterized with respect to their size and potential mechanisms of internalization (FIG. 6). Gel permeation chromatography showed that P1E28 polymer was characterized by a narrow poly dispersity and molecular weight around 5 kDa (Mn=4949, Mw=6167, Mw/Mn=1.246). In contrast to their large hydrodynamic diameter in water and in cell culture medium (FIG. 5a.2 and FIG. 14). TEM images (FIG. 6a.1) showed that most of the nanoparticles formulated by complexation of P1E28 with mRNA had a diameter up to 200 nm (FIG. 6a.2). Pre-treatment of fibroblasts with chlorpromazine, cytochalasin D and filipin inhibited Cre-mediated recombination by about 66%, 56%, and 42%, respectively, after the delivery of mRNA encoding Cre recombinase (FIG. 6b). These results indicated that the activity of P1E28 NP formulations required the involvement of different endocytic mechanisms, including clathrin-and caveolae-dependent pathways,^{[35]} as well as actin-dependent processes which are not only involved in uptake (e.g. macropinocytosis) but also in intracellular trafficking.^{[36]}

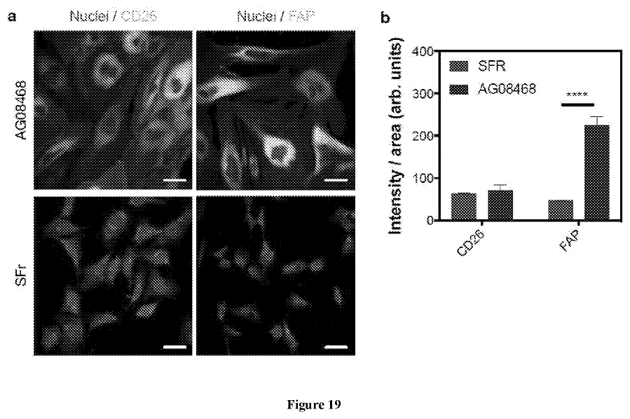

Considering the greater relevance of clathrin-dependent pathways, the enhanced activity of P1E28 in fibroblasts could be explained by receptor-mediated endocytosis. In the context of wound healing, α-SMA+activated fibroblasts derive from reticular fibroblasts overexpressing CD26,^{[17]} which are associated with fibrotic responses involving the secretion of extracellular matrix and constitute approximately 28% of the fibroblasts populating the human dermal skin.^{[37]} However, while CD26 is enriched in these fibroblasts during wound healing, it is also expressed by pro-regenerative papillary fibroblasts and keratinocytes.^{[37c, 38]} A recent study showed that CD26+reticular fibroblasts also presented high levels of FAP, whose expression is restricted to reactive fibroblasts in the skin.^{[39]} Considering that CD26 and FAP share roughly 50% of sequence homology,^{[40]} we first validated their expression in the fibroblast cell models used in this work (FIG. 19). While CD26 was expressed in both the mouse embryonic fibroblasts (SFr reporter fibroblasts) and human dermal fibroblasts (AG08468), the expression of FAP was much higher in the latter. Both receptors can be pharmacologically targeted by small molecules used as catalytic inhibitors.^{[41]} P1E28 shares some chemical features with these molecules, including the presence of aromatic (promote π-π stacking interactions with S1 pocket) and piperazine rings (mediate salt bridges with S2 pocket), in addition to the hydrogen bonds established by the polymer's carbonyl groups and amines. Even though cellular uptake of polymeric NPs may be affected by the presence of proteins and other biomolecules,^{[34]} the stability of P1E28 in medium (FIG. 14) suggests that these NPs may still mediate such interactions in vivo, in line with the observed targeting toward activated fibroblasts (FIG. 4). Therefore, the involvement of CD26 and FAP in the uptake of P1E28 polyplexes was interrogated by blocking the receptors with their respective antibodies (FIG. 6c-d). Blocking CD26 and FAP in human dermal fibroblasts significantly hampered transfection efficiency of P1E28 by 40% (p=0.0059) and 24% (p=0.0337), respectively, whereas the isotype control did not have a significant impact in transfection (p=0.1531). Similarly. Lipofectamine 2000 was not affected by receptor blocking (p=0.6243). These results were in agreement with the impaired uptake of P1E28 polyplexes after blocking CD26 (p=0.0066) and FAP (p<0.0001), whereas lipid-based DharmaFECT Duo was not affected (p=0.3845). Collectively, these results suggest that the polymeric NPs formulated by complexation of P1E28 with mRNA offer a promising solution for specific mRNA delivery to profibrotic fibroblasts by targeting CD26 and FAP receptors.

The present study reported the development of a polymeric NP-based formulation for intracellular delivery of mRNA with tropism for fibroblasts. The implication of CD26 and FAP in NP uptake pose an attractive target for mRNA delivery to fibroblasts involved in fibrotic disorders in the skin, liver, lung, and heart,^{[39b, 42]} as well as in autoimmune diseases such as rheumatoid arthritis and Crohn's disease. This formulation constitutes an alternative to NP functionalization with anti-FAP antibodies, which have been employed for modulation of the microenvironment of epithelial tumors. To the best of our knowledge, this is the first time a nanoformulation is described with tropism for fibroblasts for efficient mRNA delivery without the incorporation of specific targeting ligands. These findings may inspire the development of future formulations targeting specific cell receptors, based on the incorporation of monomers mimicking small molecule inhibitors. Although lipid nanoparticles, such as the ones used for mRNA vaccines, have shown remarkable efficiency in vivo, they offer less options in terms of chemical and physical diversity than the current polymeric formulations for cell targeting. Importantly, the identified polymers shared some chemical features resulting either in high cationic content for efficient mRNA complexation or in high buffering capacity to enable endosomal escape and mRNA release to the cytosol. Further investigation using other reporter cell types could expand this high-throughput screening methodology to identify novel formulations with specific cell tropism, and to validate the impact of polymer chemistry strategies (e.g. branching, end capping) on NP uptake and effective mRNA delivery to specific cell types.^{[11a, 11c, 11d, 45]}

### 3. Experimental Section

### 3.1. Nanoparticle Synthesis and Characterization

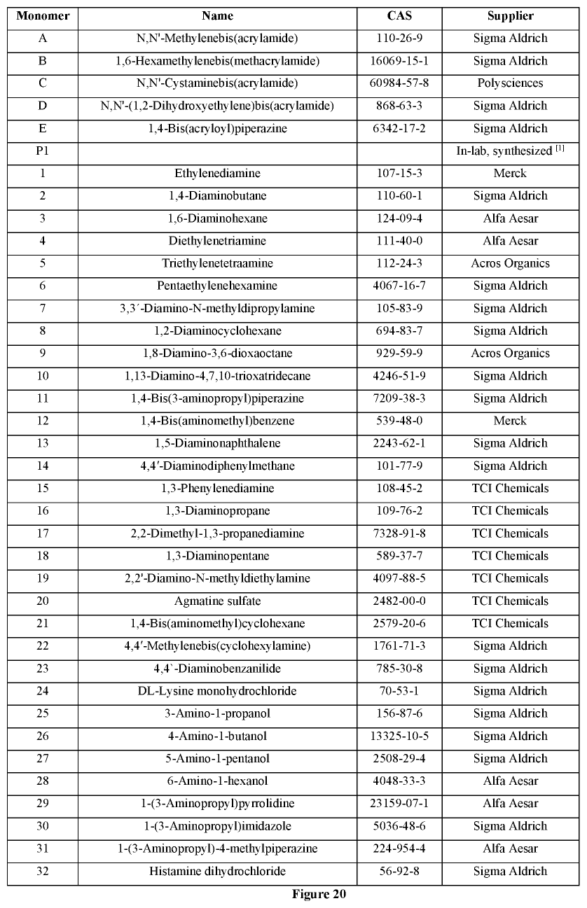

Synthesis of polymer library. Monomers used for polymer synthesis were purchased from different suppliers as described in FIG. 20. Polymers were synthesized via Michael addition reaction, as previously reported.^{[13a]} Briefly. all monomers were diluted to 1.6 M in anhydrous dimethyl sulfoxide (DMSO. Alfa Aesar), prior to the combination of the different bisacrylamides (50 μL of A-E) and amines (100 μL of 1-32) with 50 μL of diacrylate linker (P1). Reaction mixtures were added to a polypropylene 96-deep well plate (VWR), sealed with aluminum foil and kept at 60° C. for 5 days in an incubator with an orbital shaker at 250 rpm. The resulting polymers were then capped with 20 μL of the respective amine (1-32) for 3 h under the same conditions, corresponding to 10% molar excess. Polymer libraries were stored at −20° C. until further use. All polymers are described by their constituent monomers (e.g. polymer constituted by diacrylate P1, bisacrylamide A and amine 1 is abbreviated to P1A01, which is end capped by the same amine 1).

Nanoparticle size and charge. Polyplex size and ξ-potential were measured by dynamic light scattering (DLS) using a ZetaPALS Analyzer (Brookhaven Instruments Corporation). Polyplexes were prepared by mixing for 10 min the respective polymer with 1 μg Cre mRNA in milliQ ultrapure water at the optimized mass ratios, followed by dilution in ultrapure water to a final volume of 1 mL. All size measurements were performed using the same incident laser power and recorded with a scattering angle of 90°, following an equilibration time of 5 min. Each measurement corresponds to the average result of 5 runs of 1 min each. An aliquot of 100 μL of each sample was collected for nanoparticle tracking analysis (NTA). After restoring samples with the same volume of ultrapure water, ξ-potential measurements were performed by maximizing incident laser power for each sample with at least 5 runs presenting a relative residual value below 0.05. NTA was performed using a Nanosight LM10 system (Malvern Panalytical) after diluting aliquoted samples to 1 mL in ultrapure water. Samples were loaded into the chamber using a syringe pump at 10 μL/min. Five videos of 1 min each were recorded for each sample. Camera level was set at 16 and detection threshold at 4. Data analysis was performed using the NTA software (version 2.2, Malvern Panalytical).

Polymer acid-base titration. Buffering capacities of polymers were determined after dissolving 1 mg polymer in 5 mL 0.1 M sodium chloride (Fisher Scientific) and adjusting the solutions to pH 3 using 1 M hydrochloric acid (Fisher Scientific). Titration of a 5 mL sample of 0.1 M sodium chloride lacking polymer was performed as a background control. The solution pH was measured after each addition of 0.01 M sodium hydroxide (Merck) using an inoLab 720 pH meter (WTW GmbH). Buffering capacity was calculated by normalizing the amount of NaOH buffered between pH 5 and 7.5 to the total polymer mass. The derivative of the titration curves was obtained in order to determine the effective pKa, which corresponds to the pH at which the normalized buffering capacity is the highest.^{[29]}

Gel permeation chromatography. Gel permeation chromatography was performed using a size exclusion chromatography (SEC) setup from Viscotek (Viscotek TDAmax) equipped with differential viscometer (DV), right-angle laser-light scattering (RALLS, Viscotek) and refractive index (RI) detectors. The column set consisted of a Viscotek Tguard column (8 μm) followed by one Viscotek T4000 column, one Viscotek D2000 column and one Waters column WAT044232. A dual piston pump was set at a flow rate of 1 mL/min. The eluent (DMF with 0.03% LiBr) was previously filtered through a 0.2 μm filter. The system was also equipped with an on-line degasser. The tests were done at 60° C. using an Elder CH-150 heater. Before the injection (100 μL), the samples were filtered through a PTFE membrane with 0.2 μm pore. The system was calibrated with narrow PMMA standards (20-9680 Da). Molecular weight (Mn SEC) and dispersity (Ð=Mw/Mn) of the synthesized polymers were determined by TriSEC calibration using the OmniSEC software (Malvern Panalytical, version 4.6.1.354).

TEM imaging of P1E28 nanoparticles. Purified P1E28 polymer (0.5 mg mL⁻¹) was complexed with Cre. GFP and Cas9 mRNAs (with GFP sgRNA at 1:0.25 mRNA:sgRNA mass ratio) at a 1:20 mRNA:polymer mass ratio in molecular-grade, nuclease-free water. A droplet of each sample was deposited on carbon coated grids and left in a closed Petri dish at room temperature to dry for 5 h. TEM was performed using a JEOL-2100-HT electron microscope, equipped with a fast-readout “One View” 4 k×4 k CCD camera operating at 25 fps (300 fps with 512×512 pixel) and featuring drift correction. NP size distribution was carried out by manual counting on ImageJ software on several TEM images.

RNA complexation efficiency. Polyplexes were prepared by incubating polymers with 100 ng/well mRNA (encoding Cre recombinase or GFP) in nuclease-free water for 10 min, as described above. Polyplexes were then diluted to a volume of 100 μL/well in a buffer comprised of 150 mM sodium chloride (Fisher Scientific), 20 mM sodium phosphate (Sigma), 20 mM sodium acetate (Merck), and 25 mM sodium citrate (Sigma), adjusted at pH 5 or pH 7.5, as previously described.^{[30b]} Samples were prepared in triplicate at the respective pH. Standards were prepared by diluting the respective mRNA to doses ranging from 0.25 to 1 μg mL⁻¹ in the buffer set at the respective pH. Following the manufacturer's instructions, SYBR Gold (Invitrogen) was diluted 1:100 prior to the addition of 1 μL per 100 μL polyplex (final dilution 1:10000). Yellow fluorescence resulting from the hybridization of SYBR Gold to free mRNA was measured in opaque black well-plates, using a Synergy HI plate reader (Biotek) set with excitation wavelength=495 nm and emission wavelength=537 nm. Entrapment of mRNA was calculated by subtracting the amount of mRNA used to generate polyplexes (100 ng) by the amount of detected free mRNA (based on the linear calibration curve obtained from the standards).

Heparin replacement assay. Purified polymer candidates were complexed with 250 ng Cre mRNA at the optimized mRNA polymer mass ratios for 10 min. Polyplexes were then incubated for 30 min at 37° C. with 2.5-50 μg of heparin (Calbiochem), corresponding to heparin:mRNA ratio ranging 10:1-200:1. Polyplex destabilization in the presence of negatively charged heparin resulted in the release of mRNA, which was measured by agarose gel electrophoresis. Free mRNA was used as a control for electrophoretic migration upon polyplex dissociation. Agarose gels were prepared by dissolving 1% (w/v) agarose (ultrapure grade. NZYTech) in TBE 1× and staining the molten gel with 1:10000 SYBR Gold (Invitrogen) to visualize mRNA bands. Samples were loaded with 5 μL RNA loading dye 2× (Thermo Scientific), totalling a loading volume of 25 μL in each well. Electrophoresis was performed at 100 V for 30 min. Gels were imaged immediately after completing electrophoresis using a ChemiDoc XRS system (BioRad) equipped with a UV light transilluminator. Complexation studies were repeated 2-3 times for each sample.

### 3.2. In Vitro Experiments

Cell culture. Polyplex screening was performed using SFr cells derived from SC-1 mouse fibroblasts stably transduced with a retroviral SF91-loxP1-RFP-loxP2-eGFP construct^{[22]}. These cells were kindly offered by Dr Carol Stocking (University of Hamburg, Germany). SFr cells were cultured in DMEM medium (Corning) supplemented with 10% (v/v) fetal bovine serum (FBS, Life Technologies) and 0.5% (v/v) penicillin/streptomycin (VWR). Cellular tropism was validated using human dermal fibroblasts (AG08468, Coriell Institute for Medical Research; passages: 17-22) and human keratinocytes (HaCaT, passages: 32-35), both cultured in supplemented DMEM, as well as human monocytes (THP-1, passages: 7-11) cultured in supplemented RPMI 1640 (Gibco), and human umbilical vein endothelial cells (HUVECs, passages: 9-13) cultured in supplemented EGM-2 medium (Lonza). Cells were grown at 37° C. 5% CO₂ to 80-90% confluency and passaged every 3-4 days. Transfections were performed 24 hours after seeding cells in each experiment.

High-throughput screening. SFr cells (2500 cells/well) were seeded on a 96-well plate (Costar) 24 h prior to mRNA transfection. Polyplexes were prepared in sterile 96-deep well polypropylene plates (VWR) by diluting each polymer in DMSO and mixing with 50 ng/well mRNA encoding Cre recombinase, modified with 5-methoxyuridine (TriLink Biotechnologies, cat. no. L-7211), dissolved in sterile-filtered, nuclease-free water (Life Technologies) to a total volume of 10 μL/well. After incubation under agitation for 10 minutes, the generated polyplexes were diluted with 90 μL/well of serum-free DMEM for transfection. An initial optimization was performed using a selected polymer (P1C05) from a previous study,^{[13a]} a mRNA:polymer mass ratio of 1:100 (i.e. 1 μL polymer in DMSO at 5 mg mL⁻¹) was chosen for the selected dose of 50 ng/well mRNA (0.5 μg mL⁻¹), resulting in a polymer dose of 5 μg/well (50 μg mL⁻¹) for the library screening. As a positive control, 0.25 μL/well Lipofectamine® 2000 (Invitrogen) was complexed with the same mRNA dose, according to the manufacturer's instructions (volume:mass ratio=5 μL Lipofectamine μg⁻¹ mRNA). After 4 h of incubation, cells were washed to remove any remaining particles in the media and replenished with complete DMEM. After culturing for 48 h, cells were stained with Hoeschst H33342 and propidium iodide (P1), both at 1 μg mL⁻¹ (Sigma) in DMEM, and analyzed in a high-content microscope (InCell Analyzer 2200, GE Healthcare). Cellular recombination was assessed by determining the number of cells expressing GFP, using an excitation filter of 475 nm (28 nm aperture) and an emission filter 511.5 nm (23 nm aperture).

Hit validation. Identified polymer candidates were purified by dialysis in DMSO (Fisher Scientific) using pre-wetted regenerated cellulose membranes with MWCO=2 kDa (Spectrum™ Spectra/Por™ 6 Standard RC Dialysis Tubing, Fisher Scientific). Mass concentrations of purified polymers were determined after further dialysis in milliQ ultrapure water (Merck Millipore) and lyophilization by freeze-drying. SFr cells were treated with purified polymers (5-300 μg mL⁻¹) for 4 h, followed by PI staining after 48 h to evaluate polymer cytotoxicity using the InCell Analyzer 2200 microscope. Transfection of mRNA encoding EGFP, modified with 5-methoxyuridine (TriLink Biotechnologies, cat. no. L-7201), was performed under the aforementioned conditions for Cre mRNA. Different GFP mRNA:polymer ratios were tested in SFr cells using non-cytotoxic polymer concentrations. Mouse SFr fibroblasts (2500 cells/well), human AG08468 dermal fibroblasts (3000 cells/well), HaCaT keratinocytes (3000 cells/well), and HUVEC endothelial cells (3000 cells/well) were seeded 24 h prior to transfection. THP-1 monocytes (10000 cells/well) were activated with 100 ng mL⁻¹ phorbol 12-myristate 13-acetate (Sigma Aldrich) for 72 h in order to adhere to the 96-well plate, followed by 24 h of rest by washing the culture medium prior to transfection. Transfection efficiency was evaluated in mouse SFr fibroblasts, human AG08468 dermal fibroblasts, HaCaT keratinocytes, HUVEC endothelial cells and THP-1 macrophages, by quantifying the number of cells expressing GFP in a similar fashion to the aforementioned analysis of Cre-mediated recombination, using the InCell Analyzer 2200 microscope.

Delivery of Cas9 mRNA and sgRNA. SFr-GFP cells were generated in a 24-well plate after transfection of SFr cells (40000 cells/well, passage 13) with 500 ng Cre recombinase (New England Biolabs) complexed with 1.5 μL Lipofectamine R RNAiMAX (Life Technologies) in serum-free DMEM for 24 h, followed by another 24 h of culture in complete medium. The resulting SFr-GFP cells (recombination efficiency =80%) were further expanded for subsequent experiments. SFr-GFP cells were seeded in 96-well plates (3000 cells/well) 24 h prior to transfection. Polymers were first evaluated in the co-delivery of Cas9 mRNA, modified with 5-methoxyuridine (TriLink Biotechnologies, cat. no. L-7206), and a synthetic single guide RNA targeting GFP, with targeting sequence SEQ ID No 1: 5′-GGCCACAAGUUCAGCGUGUC-3′ (CRISPRevolution sgRNA EZ kit, Synthego). Polyplex stability was evaluated at different mRNA:polymer and mRNA:sgRNA ratios by agarose gel electrophoresis. Co-delivery of mRNA and sgRNA was optimized using 2 different mRNA doses (50-200 ng/well) and 4 mRNA:sgRNA ratios (1:0.25-1:2) and 3 different commercial transfection agents (volume:mass ratio=5 μL/μg mRNA), according to the respective manufacturers' instructions: Lipofectamine® 2000, Lipofectamine R, RNAiMAX and DharmaFECT™ Duo (Dharmacon, GE Healthcare). A fixed mRNA dose of 100 ng/well (1 μg mL⁻¹) was selected for similar mRNA:sgRNA optimization using polymers. Finally, mRNA:polymer mass ratios were optimized with a fixed mRNA dose of 100 ng/well and mRNA:sgRNA=1:0.25. Co-delivery of mRNA and sgRNA was compared with sequential delivery of the same mRNA dose and optimized mRNA:polymer ratio, followed by the delivery of 25 ng/well of sgRNA using Lipofectamine® 2000, 6 h or 24 h after mRNA transfection. GFP knockdown was quantified by high-content imaging 72 h after the first transfection, using the InCell Analyzer 2200 microscope. SFr-GFP cells were then expanded to 6-well plates and kept for 10 days after co-delivery of mRNA and sgRNA. Sustained GFP knockdown was measured by flow cytometry. Cells were dissociated with 0.05% (w/v) trypsin-EDTA in PBS 1× (Gibco) and centrifuged at 300 g for 3 min. Cell pellets were re-suspended in 250 μL PBS 1× and analyzed using a BD Accuri C6 flow cytometer (BD Biosciences), equipped with 488 nm and 640 nm lasers. Dead cells labelled with P1 staining and GFP expression of live cells were quantified using the 675/25 and 530/30 filters, respectively. Data were processed using FlowJo software (version X.0.7, FlowJo LLC).

Cellular uptake. Internalization of P1E28 complexed with Cas9 mRNA and GFP sgRNA was compared to the commercial agent DharmaFECT™ Duo (Dharmacon, GE Healthcare) using an ATTO550-labelled sgRNA, derived from the hybridization of an ATTO550-labelled tracrRNA and a crRNA targeting GFP (both from Integrated DNA Technologies). Briefly, both RNA molecules were mixed to a final concentration of 30 μM and incubated for 5 min at 95° C. followed by 20 min at room temperature, in order to allow hybridization of the labelled sgRNA. Particle uptake was evaluated by high content imaging and flow cytometry. For high content imaging, human dermal fibroblasts (AG08468) were seeded in a u-Slide 8-well (IBIDI) chambered coverslip (9375 cells/well). In order to compare polyplex internalization by fluorescence intensity, both P1E28 and DharmaFECT™ Duo were complexed with the same doses of Cas9 mRNA and sgRNA (150 ng/well mRNA and 37.5 ng/well; mRNA:sgRNA ratio 1:0.25). P1E28 (mRNA:polymer mass ratio=1:20; 3 μg/well) or DharmaFECT™ Duo (5 L/μg mRNA:0.75 μL/well) were incubated in the RNA mix for 10 minutes prior to transfection in serum-free DMEM for 4 h, as described above. After this time, cells were washed 3 times to remove non-internalized polyplexes and stained with DAPI (1 μg mL⁻¹; Sigma Aldrich) in PBS 1× for 5 min. ATTO550 fluorescence was evaluated using the InCell Analyzer 2200 microscope. For flow cytometry, human dermal fibroblasts (AG08468) were seeded in 12-well plates (30000 cells/well). After 24 h, both P1E28 and DharmaFECT™ Duo were complexed with a fixed dose of Cas9 mRNA (300 ng/well) and ATTO550-labelled sgRNA (75 ng/well, mRNA:sgRNA ratio=1:0.25). Briefly, P1E28 (mRNA:polymer mass ratio=1:20; 6 μg/well) or DharmaFECT™ Duo (5 μL/μg mRNA; 1.5 μL/well) were incubated in the RNA mix for 10 minutes prior to transfection in serum-free DMEM for 4 h, as described above. Cells were then washed with a heparin solution (50 μg mL⁻¹) in PBS 1×, before dissociation with 0.05% (w/v) trypsin-EDTA in PBS 1× (Gibco) and centrifugation at 300 g for 3 min. Cell pellets were re-suspended in 250 μL PBS 1× and analyzed using a BD Accuri C6 flow cytometer (BD Biosciences). Uptake of ATTO550 was quantified using the 585/40 and 530/30 filters to compensate for cell autofluorescence. Data were processed using Flow Jo software (version X.0.7, FlowJo LLC).

High-content analysis. Cell viability and GFP expression were quantified by live cell imaging using the InCell Analyzer 2200 microscope. Seven fields per well were acquired with a 20× objective using the laser autofocus method on the following channels: DAPI/Hoeschst 33342 (exposure: 0.6 s, excitation/emission: 390/435 nm), FITC/GFP (exposure: 0.4 s, excitation/emission: 475/511 nm). Texas Red/PI (exposure: 0.2 s, excitation/emission: 575/620 nm). TIFF images produced for each channel were processed using the InCell Investigator software (GE Healthcare). Cells were identified using the top-hat method in the DAPI channel (minimum area=80 μm², sensitivity=93), followed by classification of viability according to the cell nuclei. Dead cells were identified by nuclear condensation (Nuc Intensity CV<0.4) and co-localization with PI staining, determined by pseudo-segmentation in the Texas Red channel. Collar segmentation with a set radius of 3 μm in the FITC channel to identify GFP fluorescent cells, presenting cytoplasmic (perinuclear) fluorescence intensity greater than the acquired background (Reference 1 Cell/Bckg Intensity>1.03). Analysis parameters of high-content imaging were adjusted for HUVEC and THP-1 cells (sensitivity=31, Nuc Intensity CV<0.45, Cell/Bckg Intensity>1.05).

Uptake of ATTO550-labelled NPs by human dermal fibroblasts was assessed using the same software to perform a top-hat selection in the DAPI channel (minimum area=30 μm², sensitivity=70). ATTO550 fluorescent foci were quantified in a ROI delineated around the identified cell nuclei using a collar segmentation with a set radius of 5 μm, in order to account for the heterogeneous distribution of foci with different sizes and intensities. Results were expressed as the average intensity (arb. units) in the ROI and were normalized to untreated negative control.

Endocytosis inhibition. Lead polymer candidate P1E28 was complexed with Cre mRNA for transfection in SFr cells, as described above. Uptake inhibition was investigated by comparing transfection efficiency of this polyplex with cells pre-treated for 1 h with inhibitors of clathrin-mediated endocytosis (chlorpromazine hydrochloride, 10 μg mL⁻¹; Sigma Aldrich), caveolae-mediated endocytosis (filipin III, 3 μM; Sigma Aldrich) and actin-mediated processes such as macropinocytosis (cytochalasin D, 10 μM; Sigma Aldrich). These conditions were selected based on relevant studies on the cellular uptake of polyplexes and were not cytotoxic to SFr cells (data not shown).^{[32a, 35]} Polyplexes added to cells were prepared in DMEM containing the same concentration of inhibitors. Lipofectamine® 2000 was used as a positive control in the absence or presence of these inhibitors. Live GFP fluorescent cells were quantified as described above, using the InCell Analyzer 2200 microscope.

Receptor blocking for uptake and transfection studies. AG08468 human dermal fibroblasts were seeded in 96-well plates (3000 cells/well) for transfection experiments or in 12-well plates (30000 cells/well) for uptake evaluation. Cells were pre-treated for 1 h with rabbit anti-human CD26 (1:200; cat. no. 67138S. Cell Signaling Technology) or with rabbit anti-human FAP (1:200; cat. no. 66562S, Cell Signaling Technology) in serum-free DMEM, in order to block the receptors. A rabbit IgG fraction was used as an isotype control (1:1000; cat. no. X0903, Dako) to account for non-specific antibody interactions. P1E28 and Lipofectamine® 2000 were complexed with GFP mRNA for transfection, as described above. For uptake experiments, the complexes were prepared using Cas9 mRNA and ATTO550-labelled sgRNA, as described above. Particle uptake was assessed 4 h after transfection by flow cytometry. Transfection efficiency in the absence or presence of these blockers was compared 24 h after transfection by quantifying live GFP fluorescent cells, using the InCell Analyzer 2200 microscope.

Immunocytochemical analysis of fibroblasts. To assess the expression of FAP and CD26, SFr mouse reporter fibroblasts and AG08468 human dermal fibroblasts were seeded in a u-Slide 8-well (IBIDI) chambered coverslip (21250 cells/well). Cells were gently rinsed with PBS 1× 24 h after seeding and fixed with ice-cold methanol for 15 min. Cells were washed thrice with PBS for 5 min each, and permeabilized with 0.3% Triton X-100 in PBS for 10 min. After a new washing step (3×5 min with PBS), cells were incubated with blocking buffer (1% BSA and 0.3 M glycine in PBS 1×) for 1 h, followed by an overnight incubation at 4° C. with rabbit anti-human CD26 (1:200; cat. no. 67138S, Cell Signaling Technology) or with rabbit anti-human FAP (1:100; cat. no. 66562S, Cell Signaling Technology) diluted in PBS 1× containing 1% BSA and 0.3% Triton X-100. After washing with PBS (3×5 min). cells were incubated in the dark at room temperature with a goat anti-rabbit antibody labelled with AlexaFluor 488 (1:1000; cat. no. A11034; Invitrogen) diluted in PBS 1× containing 1% BSA for 1 h. After another washing step with PBS 1× (3×5 min), sections were stained with DAPI (1 μg mL⁻¹; Sigma Aldrich) for 5 minutes and washed with PBS 1× before imaging. To assess the disruption of endolysosomes, SFr mouse reporter fibroblasts were seeded in a μ-Slide 8-well (IBIDI) chambered coverslip (21250 cells/well). Cells were treated with P1E28 or DharmaFECT™ Duo complexed with a fixed dose of Cas9 mRNA (100 ng/well) and ATTO550-labelled sgRNA (25 ng/well) hydroxychloroquine in serum-free DMEM for 4 h. As a positive control, cells were treated with 60 μM hydroxychloroquine under the same conditions. Cells were washed with a heparin solution (50 μg mL⁻¹) in PBS 1× 4 h after transfection and fixed with 4% paraformaldehyde for 10 min, followed by permeabilization with 0.3% Triton X-100 and blocking with 1% BSA and 0.3 M glycine, as described above. Cells were incubated for 1 h with the rat anti-galectin 9 (1:100; cat. no. 137901, BioLegend) diluted in PBS 1× containing 1% BSA, followed by a washing step with PBS to remove the primary antibody. Cells were then incubated with the secondary chicken anti-rat IgG Alexa Fluor 488 (1:1000; cat. no. A21470; Invitrogen) diluted in PBS 1× containing 1% BSA for 1 h, and the nuclei stained with DAPI (1 μg mL⁻¹; Sigma Aldrich) for 5 min. For both experiments, cells were imaged using a Zeiss LSM 710 confocal microscope, equipped with an Apochromat 40×/1.4 objective. Images were analyzed in ImageJ (version 1.51, NIH) to count individual galectin-9 foci and measure fluorescence intensity levels of CD26 and FAP in each cell using the Analyze Particles built-in function.

### 3.3. In Vivo Experiments

Experimental animals. Nineteen-week-old male and female (22-24 g) R26-tdTomato reporter mice (C57BL/6 mice bearing a loxP-flanked STOP cassette impeding tdTomato expression) were used in the present study, in accordance with the ARRIVE guidelines, and after ethical approval from the Ethics Committee of the Faculty of Medicine of the University of Coimbra (ORBEA_159_2017/05052017) and the Portuguese National Authority for Animal Health (DGAV project reference: 015433_0421/000/000/2017). Animals were maintained in groups of 5 with free access to water and food, under a regular 12 h light/dark cycle at a temperature of 19-22° C. and relative humidity of 45-65%.

Wound induction. Animals were separated in individual cages 24 h before induction of skin wounds. After anesthetizing by inhalation of 3% isoflurane in oxygen flowing at 2 L/min, the dorsal skin of each mouse was shaved and disinfected using iodopovidone (Betadine R)). Two full-thickness excision wounds (6 mm diameter) were inflicted 5 cm apart using a sterile biopsy punch in each animal. In order to minimize distress, analgesia (buprenorphine, 0.1mg kg⁻¹) was administered subcutaneously 30 min before wound induction, and every 6-8 h up to 48 h post wounding.

In vivo mRNA delivery. At day 5 post wounding, P1E28 or Lipofectamine® 2000 (both n=6 mice; 3 male+3 female) were complexed with 7 μg Cre mRNA (0.3 mg kg⁻¹ mouse) for 10 minutes, prior to dilution in serum-free DMEM to a final volume of 120 μL/mouse. Each treatment was administered by intradermal injection across 4 different locations around the wound (30 μL/injection). Two male mice (vehicle controls) were administered with the same volume containing only serum-free DMEM. All animals were monitored daily for 12 days after wound induction. Wound healing was monitored by measuring the wound area over time. Mice were sacrificed 7 days post injection by cervical dislocation and skin samples were harvested for immunohistochemical analysis.

Immunohistochemical analysis of skin wounds. Skin samples were dissected with an excess area around the respective wound and fixed in 10% neutral buffered formalin overnight at 4° C. with the subcutaneous tissue interfacing a small piece of cardboard to keep the sample flat. Fixed skin samples were embedded in optical cutting temperature (OCT) and snap-frozen for cryo-sectioning. Longitudinal sections were obtained using a Leica CM3050S cryotome set at a thickness of 10 μm, and stored at −80° C. until further processing. Skin sections were gently rinsed with PBS 1× to remove excess OCT, and permeabilized with 0.3% Triton X-100 in PBS for 10 minutes. Sections were washed thrice with PBS for 5 min each, followed by an 1 h incubation in blocking buffer (5% BSA and 0.3 M glycine in PBS 1×) to block non-specific interactions with proteins and unreacted aldehydes derived from fixation. After removing blocking solution, sections were incubated overnight at 4° C. with primary antibodies diluted in PBS 1× containing 1% BSA. After washing with PBS 1× (3×5 min), sections were incubated in the dark at room temperature with the respective secondary antibody diluted in PBS 1× containing 1% BSA for 1 h. After another washing step with PBS 1× (3×5 min), sections were stained with DAPI (1 μg mL⁻¹; Sigma Aldrich) for 5 minutes and blot dried before mounting with fluorescence mounting medium (Dako). Sections were imaged using the InCell Analyzer 2200 microscope, set with a 20× objective. Images from entire sections were acquired and stitched together using the InCell Developer software (GE Healthcare). Skin samples obtained from the vehicle control were imaged in order to establish threshold levels for tissue autofluorescence. Cre-mediated recombination was quantified in all wounds (n=14) using ImageJ (version 1.51, NIH) to count individual cells with fluorescence intensity levels above the aforementioned thresholds. Fibroblasts were stained using rabbit anti-α-SMA (1/100; cat. no. ab5694; Abcam), followed by anti-rabbit labelled with Cy3 (1/250; cat. no. 111-165-144; Jackson ImmunoResearch Europe Ltd). Endothelial cells were detected using goat anti-CD31 (1/40; cat. no. AF3628; R&D Systems) followed by donkey anti-goat labelled with AlexaFluor 488 (1/1000; cat. no. A11055; Invitrogen). Keratinocytes were identified using rabbit anti-keratin 14 (1/1000; cat. no. 905301; BioLegend) or chicken anti-keratin 5 (1/200; cat. no. 905901; BioLegend), followed by goat anti-rabbit labelled with AlexaFluor 488 (1/1000; cat. no. A11034; Invitrogen) or anti-chicken labelled with AlexaFluor 488 (1/1000; cat. no. A11039; Invitrogen), respectively. Macrophages were labelled using rat anti-CD68 (1/100; cat. no. MCA1957; BioRad) followed by anti-rat labelled with PE (1/500; cat. no. F0105B; R&D Systems). Co-localization with the identified cell types was quantified in independent skin samples exhibiting Cre-mediated recombination (i.e. tdTomato fluorescence) using the JACOP plugin on ImageJ, after adjusting fluorescence intensity levels to minimize autofluorescence artifacts. Co-localization of each cell marker with the tdTomato signal was expressed as the Manders' overlap coefficient×100%.

Statistical analysis. All experiments were analyzed using GraphPad Prism software (version 7, GraphPad Inc.). Data were analyzed using 1- or 2-way ANOVA tests with post hoc multiple comparisons test, provided statistical significance was obtained when p<0.05. Multivariable analysis of polymer characteristics on its transfection efficiency was performed using JMP software (version 13, SAS). The data were fitted to a standard least squares model. set to effect leverage considering first-order effects.

### REFERENCES

[1] S. L. Ginn, et al., J Gene Med 2018, 20, e3015.
[2] a) K. A. Hajj, K. A. Whitehead, Nat Rev Mater 2017, 2, 17056; b) J. C. Kaczmarek, et al., Genome Med 2017, 9, 60.
[3] a) J. A. Doudna, Nature 2020, 578, 229-236; b) H. Yin, et al., Nat Rev Drug Discov 2017, 16, 387-399.
[4] H. Yin, R. L. Kanasty, A. A. Eltoukhy, A. J. Vegas, J. R. Dorkin, D. G. Anderson, Nature Reviews Genetics 2014, 15, 541-555.
[5] A. Akinc, et al., Nat Nanotechnol 2019, 14, 1084-1087.
[6] a) A. J. Barbier, et al., Nat Biotechnol 2022, 40, 840-854; b) M. P. Lokugamage, et al., Adv Mater 2020, 32, 1904905.
[7] a) J. Liu, et al., Adv Mater 2019; b) H. Yin, et al., Nature Biotechnol 2016, 34, 328-333.
[8] N. Veiga, et al., Nat Commun 2018, 9, 4493.
[9] Z. Cheng, et al., Science 2012, 338, 903-910.

1. A polymeric nanoparticle formulation for targeted delivery of mRNA to cells comprising a polymer made of divinyl monomers bearing an aromatic ring, piperazine-containing divinyl monomers, and amino alcohol monomers, wherein the polymer is complexed with mRNA to form the nanoparticles.

2. The polymeric nanoparticle formulation according to claim 1, wherein the divinyl monomer bearing an aromatic ring is selected from (2-nitro-1,3-phenylene)bis(methylene) diacrylate or (1,3-phenylene)bis(methylene)diacrylate.

3. The polymeric nanoparticle formulation according to claim 1, wherein the piperazine-containing divinyl monomer is selected from 1,4-bis(acryloyl)piperazine.

4. The polymeric nanoparticle formulation according to claim 1, wherein the amino alcohol monomers are selected from 3-amino-1-propanol, 4-amino-1-butanol, 5-amino-1-pentanol, 6-amino-1-hexanol, 7-amino-1-heptanol, 8-amino-1-octanol, 9-amino-1-nonanol, or 10-amino-1-decanol.

5. The polymeric nanoparticle formulation according to claim 1, wherein the molar ratio per repeating unit of the polymer is 50% divinyl monomers:50% amino alcohol.

6. The polymeric nanoparticle formulation according to claim 1, wherein the molar ratio per repeating unit of the polymer of any of the divinyl monomers varies between 5% and 45%.

7. The polymeric nanoparticle formulation according to claim 1, wherein the mass ratio of mRNA:polymer is between 1:5 and 1:200.

8. The polymeric nanoparticle formulation according to claim 1, wherein the mass ratio of mRNA:polymer is 1:100.

9. The polymeric nanoparticle formulation according to claim 1, wherein the nanoparticles have an average particle size between 200 and 600 nm.

10. The polymeric nanoparticle formulation according to claim 1, wherein the nanoparticles have a zeta potential between +5 and +25 mV.

11. A method for delivery of mRNA to fibroblasts in skin, heart, liver, lung, synovial joints, or the central nervous system comprising administering the polymeric nanoparticle formulation of claim 1 to skin, heart, liver, lung, synovial joints, or the central nervous system of a patient in need thereof.

12. A method of treating fibrotic disorders in the skin, liver, lung, or heart, autoimmune diseases, or oncological disorders comprising using the polymeric nanoparticle formulation of claim 1 to cause modulation of fibroblasts to remodel extracellular matrix in the skin and the microenvironment of epithelial tumors; or in cellular reprogramming of fibroblasts in vivo and ex vivo.
